SEQUENCE LISTING (174 sequences)

SEQ ID NO: 1 - DNA, 44 bp, synthetic construct
  source 1..44: mol_type = other DNA
acccaactgaatggagctatattcagtatggaaagaataaaaga

SEQ ID NO: 2 - DNA, 39 bp, synthetic construct
  source 1..39: mol_type = other DNA
acccaactgaatggagcacggtccattacccaaaagtgt

SEQ ID NO: 3 - DNA, 40 bp, synthetic construct
  source 1..40: mol_type = other DNA
acccaactgaatggagcgtgcggaaaacaagatttctccc

SEQ ID NO: 4 - DNA, 39 bp, synthetic construct
  source 1..39: mol_type = other DNA
acccaactgaatggagctcatctttcagctttggtggct

SEQ ID NO: 5 - DNA, 39 bp, synthetic construct
  source 1..39: mol_type = other DNA
acccaactgaatggagccagagcaaatcagcggttgaac

SEQ ID NO: 6 - DNA, 39 bp, synthetic construct
  source 1..39: mol_type = other DNA
acccaactgaatggagcatgatgtgggagattaacggcc

SEQ ID NO: 7 - DNA, 38 bp, synthetic construct
  source 1..38: mol_type = other DNA
acccaactgaatggagcccaccgaagcaaagcagaatg

SEQ ID NO: 8 - DNA, 41 bp, synthetic construct
  source 1..41: mol_type = other DNA
acccaactgaatggagccaagaccatttgaatggatgtcaa

SEQ ID NO: 9 - DNA, 39 bp, synthetic construct
  source 1..39: mol_type = other DNA
acccaactgaatggagcttgatggaccactacctgagga

SEQ ID NO: 10 - DNA, 42 bp, synthetic construct
  source 1..42: mol_type = other DNA
acccaactgaatggagctcaatggataaggaggaaatggaga

SEQ ID NO: 11 - DNA, 40 bp, synthetic construct
  source 1..40: mol_type = other DNA
acccaactgaatggagcccaaactggcaaatgttgtgaga

SEQ ID NO: 12 - DNA, 46 bp, synthetic construct
  source 1..46: mol_type = other DNA
acccaactgaatggagccaatgaatcaacaaggaagaaaattgaga

SEQ ID NO: 13 - DNA, 42 bp, synthetic construct
  source 1..42: mol_type = other DNA
acccaactgaatggagcacagggacatttgaattcactagct

SEQ ID NO: 14 - DNA, 42 bp, synthetic construct
  source 1..42: mol_type = other DNA
acccaactgaatggagccaggattattggtatcagatggggg

SEQ ID NO: 15 - DNA, 43 bp, synthetic construct
  source 1..43: mol_type = other DNA
acccaactgaatggagcacttgttcgagaaatttttccctagt

SEQ ID NO: 16 - DNA, 39 bp, synthetic construct
  source 1..39: mol_type = other DNA
acccaactgaatggagcgcttcaacccgatgattgtcga

SEQ ID NO: 17 - DNA, 42 bp, synthetic construct
  source 1..42: mol_type = other DNA
acccaactgaatggagcaaatcagagaacacacacattcaca

SEQ ID NO: 18 - DNA, 39 bp, synthetic construct
  source 1..39: mol_type = other DNA
acccaactgaatggagcgggcaagctttctcaaatgtcc

SEQ ID NO: 19 - DNA, 44 bp, synthetic construct
  source 1..44: mol_type = other DNA
acccaactgaatggagctgaaaatgaggagaagattccaagaac

SEQ ID NO: 20 - DNA, 39 bp, synthetic construct
  source 1..39: mol_type = other DNA
acccaactgaatggagcgtcccattgcagagctactgaa

SEQ ID NO: 21 - DNA, 39 bp, synthetic construct
  source 1..39: mol_type = other DNA
acccaactgaatggagcgccataggccaaatttcaaggc

SEQ ID NO: 22 - DNA, 39 bp, synthetic construct
  source 1..39: mol_type = other DNA
acccaactgaatggagcgcagagtcaagaaaactgctcc

SEQ ID NO: 23 - DNA, 44 bp, synthetic construct
  source 1..44: mol_type = other DNA
acccaactgaatggagctaatgtctattaaccatgaagactatc

SEQ ID NO: 24 - DNA, 39 bp, synthetic construct
  source 1..39: mol_type = other DNA
acccaactgaatggagctccggcacactggagtttaaaa

SEQ ID NO: 25 - DNA, 43 bp, synthetic construct
  source 1..43: mol_type = other DNA
acccaactgaatggagctccctagcagaataagcatctattgg

SEQ ID NO: 26 - DNA, 39 bp, synthetic construct
  source 1..39: mol_type = other DNA
acccaactgaatggagcacaaaccagaggcatatttggc

SEQ ID NO: 27 - DNA, 39 bp, synthetic construct
  source 1..39: mol_type = other DNA
acccaactgaatggagcttgccctggagaaccaacatac

SEQ ID NO: 28 - DNA, 40 bp, synthetic construct
  source 1..40: mol_type = other DNA
acccaactgaatggagcgtagataatcactcactgagtga

SEQ ID NO: 29 - DNA, 47 bp, synthetic construct
  source 1..47: mol_type = other DNA
acccaactgaatggagctgatgaaagaagaaataaatacctggaaga

SEQ ID NO: 30 - DNA, 39 bp, synthetic construct
  source 1..39: mol_type = other DNA
acccaactgaatggagcaggtgctgcagtcaaaggaatc

SEQ ID NO: 31 - DNA, 39 bp, synthetic construct
  source 1..39: mol_type = other DNA
acccaactgaatggagccctgcagtatccagtggttacg

SEQ ID NO: 32 - DNA, 44 bp, synthetic construct
  source 1..44: mol_type = other DNA
acccaactgaatggagctcaactagaggagtacaaattgcttca

SEQ ID NO: 33 - DNA, 40 bp, synthetic construct
  source 1..40: mol_type = other DNA
acccaactgaatggagccgattggctctgtttctctcaca

SEQ ID NO: 34 - DNA, 42 bp, synthetic construct
  source 1..42: mol_type = other DNA
acccaactgaatggagcagtgtatttgaccaacaccaccata

SEQ ID NO: 35 - DNA, 39 bp, synthetic construct
  source 1..39: mol_type = other DNA
acccaactgaatggagcgtgttcctttccatctggggac

SEQ ID NO: 36 - DNA, 39 bp, synthetic construct
  source 1..39: mol_type = other DNA
acccaactgaatggagcgaagtgctcagcatgtcgaaga

SEQ ID NO: 37 - DNA, 39 bp, synthetic construct
  source 1..39: mol_type = other DNA
acccaactgaatggagctcaaagtcgttgaaggctggtc

SEQ ID NO: 38 - DNA, 42 bp, synthetic construct
  source 1..42: mol_type = other DNA
acccaactgaatggagctagatattgaaagatgatcttaccg

SEQ ID NO: 39 - DNA, 40 bp, synthetic construct
  source 1..40: mol_type = other DNA
acccaactgaatggagctggagacccaaataacatggaca

SEQ ID NO: 40 - DNA, 38 bp, synthetic construct
  source 1..38: mol_type = other DNA
acccaactgaatggagcgcaaatggctggctcaagtga

SEQ ID NO: 41 - DNA, 43 bp, synthetic construct
  source 1..43: mol_type = other DNA
acccaactgaatggagcgtgtataagagacataatggattcca

SEQ ID NO: 42 - DNA, 44 bp, synthetic construct
  source 1..44: mol_type = other DNA
acccaactgaatggagcgcaaatcgtagaaaagattctgaaagg

SEQ ID NO: 43 - DNA, 39 bp, synthetic construct
  source 1..39: mol_type = other DNA
acccaactgaatggagcattgctaagggctttcaccgaa

SEQ ID NO: 44 - DNA, 41 bp, synthetic construct
  source 1..41: mol_type = other DNA
acgcacttgacttgtcttcaaaatggacagggccaaaggtt

SEQ ID NO: 45 - DNA, 41 bp, synthetic construct
  source 1..41: mol_type = other DNA
acgcacttgacttgtcttcttcccaacacgttccttgagtc

SEQ ID NO: 46 - DNA, 43 bp, synthetic construct
  source 1..43: mol_type = other DNA
acgcacttgacttgtcttctgcctgtaagaacctcttcttctc

SEQ ID NO: 47 - DNA, 42 bp, synthetic construct
  source 1..42: mol_type = other DNA
acgcacttgacttgtcttccgatgtgttcaactccccaattt

SEQ ID NO: 48 - DNA, 41 bp, synthetic construct
  source 1..41: mol_type = other DNA
acgcacttgacttgtcttcttgacagcttcccaattcctga

SEQ ID NO: 49 - DNA, 44 bp, synthetic construct
  source 1..44: mol_type = other DNA
acgcacttgacttgtcttcagccttttagtggtcttgttgtagt

SEQ ID NO: 50 - DNA, 48 bp, synthetic construct
  source 1..48: mol_type = other DNA
acgcacttgacttgtcttctttttaaactattcagtattaattgatgg

SEQ ID NO: 51 - DNA, 43 bp, synthetic construct
  source 1..43: mol_type = other DNA
acgcacttgacttgtcttcggattcttcaagaaaggccatagc

SEQ ID NO: 52 - DNA, 44 bp, synthetic construct
  source 1..44: mol_type = other DNA
acgcacttgacttgtcttcgttctttgtgtgaccattttcttgg

SEQ ID NO: 53 - DNA, 42 bp, synthetic construct
  source 1..42: mol_type = other DNA
acgcacttgacttgtcttctccacttagtgttgtctccagtg

SEQ ID NO: 54 - DNA, 41 bp, synthetic construct
  source 1..41: mol_type = other DNA
acgcacttgacttgtcttcacatgcccatcatcattccagg

SEQ ID NO: 55 - DNA, 44 bp, synthetic construct
  source 1..44: mol_type = other DNA
acgcacttgacttgtcttctcgtttattccagacactccaaaac

SEQ ID NO: 56 - DNA, 41 bp, synthetic construct
  source 1..41: mol_type = other DNA
acgcacttgacttgtcttcccccgataattgtcgtccatca

SEQ ID NO: 57 - DNA, 45 bp, synthetic construct
  source 1..45: mol_type = other DNA
acgcacttgacttgtcttccattttttcatgaaggacaagctaaa

SEQ ID NO: 58 - DNA, 43 bp, synthetic construct
  source 1..43: mol_type = other DNA
acgcacttgacttgtcttctcgccttgttcattgatgaaatgg

SEQ ID NO: 59 - DNA, 41 bp, synthetic construct
  source 1..41: mol_type = other DNA
acgcacttgacttgtcttctcctcgtcgagagtgtagtctg

SEQ ID NO: 60 - DNA, 41 bp, synthetic construct
  source 1..41: mol_type = other DNA
acgcacttgacttgtcttctccactgggaagtttgattggt

SEQ ID NO: 61 - DNA, 41 bp, synthetic construct
  source 1..41: mol_type = other DNA
acgcacttgacttgtcttctctaccttctctggtgccatgt

SEQ ID NO: 62 - DNA, 41 bp, synthetic construct
  source 1..41: mol_type = other DNA
acgcacttgacttgtcttcagttgaaaatcgtccattgccg

SEQ ID NO: 63 - DNA, 41 bp, synthetic construct
  source 1..41: mol_type = other DNA
acgcacttgacttgtcttccaacgtctcatctccattcccc

SEQ ID NO: 64 - DNA, 42 bp, synthetic construct
  source 1..42: mol_type = other DNA
acgcacttgacttgtcttctagcttttttggacagtacggat

SEQ ID NO: 65 - DNA, 44 bp, synthetic construct
  source 1..44: mol_type = other DNA
acgcacttgacttgtcttctctgaaccaactcagtagcattagt

SEQ ID NO: 66 - DNA, 46 bp, synthetic construct
  source 1..46: mol_type = other DNA
acgcacttgacttgtcttcaaaactactactagatccccttatgca

SEQ ID NO: 67 - DNA, 42 bp, synthetic construct
  source 1..42: mol_type = other DNA
acgcacttgacttgtcttcggagcaattagattccctgtgct

SEQ ID NO: 68 - DNA, 41 bp, synthetic construct
  source 1..41: mol_type = other DNA
acgcacttgacttgtcttctgatgcctgaaaccgtaccaac

SEQ ID NO: 69 - DNA, 41 bp, synthetic construct
  source 1..41: mol_type = other DNA
acgcacttgacttgtcttctcagcattttccctcagttgct

SEQ ID NO: 70 - DNA, 44 bp, synthetic construct
  source 1..44: mol_type = other DNA
acgcacttgacttgtcttcgtttttaattaatgcactcaaatgc

SEQ ID NO: 71 - DNA, 41 bp, synthetic construct
  source 1..41: mol_type = other DNA
acgcacttgacttgtcttcgactcttctgtatatgggcccc

SEQ ID NO: 72 - DNA, 41 bp, synthetic construct
  source 1..41: mol_type = other DNA
acgcacttgacttgtcttcaatttcgatcgttgatcccccg

SEQ ID NO: 73 - DNA, 46 bp, synthetic construct
  source 1..46: mol_type = other DNA
acgcacttgacttgtcttcggctattttgaagtagtttgaaagggt

SEQ ID NO: 74 - DNA, 41 bp, synthetic construct
  source 1..41: mol_type = other DNA
acgcacttgacttgtcttcaaaacgtaggttgcacactggt

SEQ ID NO: 75 - DNA, 41 bp, synthetic construct
  source 1..41: mol_type = other DNA
acgcacttgacttgtcttcggtattgtttccttaattgtcg

SEQ ID NO: 76 - DNA, 41 bp, synthetic construct
  source 1..41: mol_type = other DNA
acgcacttgacttgtcttcccacattgcggttttgaccaat

SEQ ID NO: 77 - DNA, 41 bp, synthetic construct
  source 1..41: mol_type = other DNA
acgcacttgacttgtcttccccgttatacaaacatgcagcc

SEQ ID NO: 78 - DNA, 42 bp, synthetic construct
  source 1..42: mol_type = other DNA
acgcacttgacttgtcttcggatcctttccagttgtctctgc

SEQ ID NO: 79 - DNA, 44 bp, synthetic construct
  source 1..44: mol_type = other DNA
acgcacttgacttgtcttcgagtttgtttctataattgcgaaag

SEQ ID NO: 80 - DNA, 41 bp, synthetic construct
  source 1..41: mol_type = other DNA
acgcacttgacttgtcttcatcttcaagtctctgcgcgatc

SEQ ID NO: 81 - DNA, 41 bp, synthetic construct
  source 1..41: mol_type = other DNA
acgcacttgacttgtcttctgagagctatttctttggcccc

SEQ ID NO: 82 - DNA, 41 bp, synthetic construct
  source 1..41: mol_type = other DNA
acgcacttgacttgtcttcactaggatgagtcccaatggct

SEQ ID NO: 83 - DNA, 44 bp, synthetic construct
  source 1..44: mol_type = other DNA
acgcacttgacttgtcttcgtagttttttactccaattctatgc

SEQ ID NO: 84 - DNA, 43 bp, synthetic construct
  source 1..43: mol_type = other DNA
acgcacttgacttgtcttcagtcatgtcagttatgtatcgcga

SEQ ID NO: 85 - DNA, 47 bp, synthetic construct
  source 1..47: mol_type = other DNA
acgcacttgacttgtcttctgtagatttttagagactcgaactgtgt

SEQ ID NO: 86 - DNA, 46 bp, synthetic construct
  source 1..46: mol_type = other DNA
acgcacttgacttgtcttcgtgtgttttttatcattaaataagctg

SEQ ID NO: 87 - DNA, 27 bp, synthetic construct
  source 1..27: mol_type = other DNA
tatattcagtatggaaagaataaaaga

SEQ ID NO: 88 - DNA, 22 bp, synthetic construct
  source 1..22: mol_type = other DNA
aaaatggacagggccaaaggtt

SEQ ID NO: 89 - DNA, 22 bp, synthetic construct
  source 1..22: mol_type = other DNA
acggtccattacccaaaagtgt

SEQ ID NO: 90 - DNA, 22 bp, synthetic construct
  source 1..22: mol_type = other DNA
ttcccaacacgttccttgagtc

SEQ ID NO: 91 - DNA, 23 bp, synthetic construct
  source 1..23: mol_type = other DNA
gtgcggaaaacaagatttctccc

SEQ ID NO: 92 - DNA, 24 bp, synthetic construct
  source 1..24: mol_type = other DNA
tgcctgtaagaacctcttcttctc

SEQ ID NO: 93 - DNA, 22 bp, synthetic construct
  source 1..22: mol_type = other DNA
tcatctttcagctttggtggct

SEQ ID NO: 94 - DNA, 24 bp, synthetic construct
  source 1..24: mol_type = other DNA
caagaccatttgaatggatgtcaa

SEQ ID NO: 95 - DNA, 22 bp, synthetic construct
  source 1..22: mol_type = other DNA
cagagcaaatcagcggttgaac

SEQ ID NO: 96 - DNA, 22 bp, synthetic construct
  source 1..22: mol_type = other DNA
ttgacagcttcccaattcctga

SEQ ID NO: 97 - DNA, 22 bp, synthetic construct
  source 1..22: mol_type = other DNA
atgatgtgggagattaacggcc

SEQ ID NO: 98 - DNA, 25 bp, synthetic construct
  source 1..25: mol_type = other DNA
agccttttagtggtcttgttgtagt

SEQ ID NO: 99 - DNA, 21 bp, synthetic construct
  source 1..21: mol_type = other DNA
ccaccgaagcaaagcagaatg

SEQ ID NO: 100 - DNA, 23 bp, synthetic construct
  source 1..23: mol_type = other DNA
agcattaattgatggccatccga

SEQ ID NO: 101 - DNA, 22 bp, synthetic construct
  source 1..22: mol_type = other DNA
cgtggtgttggtaatgaaacgg

SEQ ID NO: 102 - DNA, 22 bp, synthetic construct
  source 1..22: mol_type = other DNA
gcttcaacccgatgattgtcga

SEQ ID NO: 103 - DNA, 22 bp, synthetic construct
  source 1..22: mol_type = other DNA
ttgatggaccactacctgagga

SEQ ID NO: 104 - DNA, 25 bp, synthetic construct
  source 1..25: mol_type = other DNA
gttctttgtgtgaccattttcttgg

SEQ ID NO: 105 - DNA, 25 bp, synthetic construct
  source 1..25: mol_type = other DNA
tcaatggataaggaggaaatggaga

SEQ ID NO: 106 - DNA, 23 bp, synthetic construct
  source 1..23: mol_type = other DNA
tccacttagtgttgtctccagtg

SEQ ID NO: 107 - DNA, 23 bp, synthetic construct
  source 1..23: mol_type = other DNA
ccaaactggcaaatgttgtgaga

SEQ ID NO: 108 - DNA, 22 bp, synthetic construct
  source 1..22: mol_type = other DNA
acatgcccatcatcattccagg

SEQ ID NO: 109 - DNA, 27 bp, synthetic construct
  source 1..27: mol_type = other DNA
taatgtctattaaccatgaagactatc

SEQ ID NO: 110 - DNA, 25 bp, synthetic construct
  source 1..25: mol_type = other DNA
tcgtttattccagacactccaaaac

SEQ ID NO: 111 - DNA, 25 bp, synthetic construct
  source 1..25: mol_type = other DNA
acagggacatttgaattcactagct

SEQ ID NO: 112 - DNA, 22 bp, synthetic construct
  source 1..22: mol_type = other DNA
ccccgataattgtcgtccatca

SEQ ID NO: 113 - DNA, 25 bp, synthetic construct
  source 1..25: mol_type = other DNA
caggattattggtatcagatggggg

SEQ ID NO: 114 - DNA, 23 bp, synthetic construct
  source 1..23: mol_type = other DNA
gtagataatcactcactgagtga

SEQ ID NO: 115 - DNA, 26 bp, synthetic construct
  source 1..26: mol_type = other DNA
acttgttcgagaaatttttccctagt

SEQ ID NO: 116 - DNA, 24 bp, synthetic construct
  source 1..24: mol_type = other DNA
tcgccttgttcattgatgaaatgg

SEQ ID NO: 117 - DNA, 24 bp, synthetic construct
  source 1..24: mol_type = other DNA
ttgaaaccaacaaatttgcagcaa

SEQ ID NO: 118 - DNA, 22 bp, synthetic construct
  source 1..22: mol_type = other DNA
tcctcgtcgagagtgtagtctg

SEQ ID NO: 119 - DNA, 23 bp, synthetic construct
  source 1..23: mol_type = other DNA
cgattggctctgtttctctcaca

SEQ ID NO: 120 - DNA, 22 bp, synthetic construct
  source 1..22: mol_type = other DNA
tccactgggaagtttgattggt

SEQ ID NO: 121 - DNA, 22 bp, synthetic construct
  source 1..22: mol_type = other DNA
gggcaagctttctcaaatgtcc

SEQ ID NO: 122 - DNA, 22 bp, synthetic construct
  source 1..22: mol_type = other DNA
tctaccttctctggtgccatgt

SEQ ID NO: 123 - DNA, 27 bp, synthetic construct
  source 1..27: mol_type = other DNA
tgaaaatgaggagaagattccaagaac

SEQ ID NO: 124 - DNA, 25 bp, synthetic construct
  source 1..25: mol_type = other DNA
tagatattgaaagatgatcttaccg

SEQ ID NO: 125 - DNA, 22 bp, synthetic construct
  source 1..22: mol_type = other DNA
gtcccattgcagagctactgaa

SEQ ID NO: 126 - DNA, 22 bp, synthetic construct
  source 1..22: mol_type = other DNA
caacgtctcatctccattcccc

SEQ ID NO: 127 - DNA, 26 bp, synthetic construct
  source 1..26: mol_type = other DNA
gtgtataagagacataatggattcca

SEQ ID NO: 128 - DNA, 24 bp, synthetic construct
  source 1..24: mol_type = other DNA
cctcaattgcttcatatagccccc

SEQ ID NO: 129 - DNA, 22 bp, synthetic construct
  source 1..22: mol_type = other DNA
gcagagtcaagaaaactgctcc

SEQ ID NO: 130 - DNA, 25 bp, synthetic construct
  source 1..25: mol_type = other DNA
tctgaaccaactcagtagcattagt

SEQ ID NO: 131 - DNA, 22 bp, synthetic construct
  source 1..22: mol_type = other DNA
caccatgcagtaccaaacggaa

SEQ ID NO: 132 - DNA, 27 bp, synthetic construct
  source 1..27: mol_type = other DNA
aaaactactactagatccccttatgca

SEQ ID NO: 133 - DNA, 22 bp, synthetic construct
  source 1..22: mol_type = other DNA
tccggcacactggagtttaaaa

SEQ ID NO: 134 - DNA, 23 bp, synthetic construct
  source 1..23: mol_type = other DNA
ggagcaattagattccctgtgct

SEQ ID NO: 135 - DNA, 26 bp, synthetic construct
  source 1..26: mol_type = other DNA
tccctagcagaataagcatctattgg

SEQ ID NO: 136 - DNA, 29 bp, synthetic construct
  source 1..29: mol_type = other DNA
tttttaaactattcagtattaattgatgg

SEQ ID NO: 137 - DNA, 22 bp, synthetic construct
  source 1..22: mol_type = other DNA
acaaaccagaggcatatttggc

SEQ ID NO: 138 - DNA, 22 bp, synthetic construct
  source 1..22: mol_type = other DNA
tcagcattttccctcagttgct

SEQ ID NO: 139 - DNA, 22 bp, synthetic construct
  source 1..22: mol_type = other DNA
ttgccctggagaaccaacatac

SEQ ID NO: 140 - DNA, 29 bp, synthetic construct
  source 1..29: mol_type = other DNA
actcagtgagtgattatctacgtttttaa

SEQ ID NO: 141 - DNA, 22 bp, synthetic construct
  source 1..22: mol_type = other DNA
ttgttggggttcatcatgtggg

SEQ ID NO: 142 - DNA, 22 bp, synthetic construct
  source 1..22: mol_type = other DNA
gactcttctgtatatgggcccc

SEQ ID NO: 143 - DNA, 26 bp, synthetic construct
  source 1..26: mol_type = other DNA
cattttttcatgaaggacaagctaaa

SEQ ID NO: 144 - DNA, 22 bp, synthetic construct
  source 1..22: mol_type = other DNA
aatttcgatcgttgatcccccg

SEQ ID NO: 145 - DNA, 22 bp, synthetic construct
  source 1..22: mol_type = other DNA
aggtgctgcagtcaaaggaatc

SEQ ID NO: 146 - DNA, 27 bp, synthetic construct
  source 1..27: mol_type = other DNA
ggctattttgaagtagtttgaaagggt

SEQ ID NO: 147 - DNA, 22 bp, synthetic construct
  source 1..22: mol_type = other DNA
cctgcagtatccagtggttacg

SEQ ID NO: 148 - DNA, 22 bp, synthetic construct
  source 1..22: mol_type = other DNA
aaaacgtaggttgcacactggt

SEQ ID NO: 149 - DNA, 27 bp, synthetic construct
  source 1..27: mol_type = other DNA
tcaactagaggagtacaaattgcttca

SEQ ID NO: 150 - DNA, 23 bp, synthetic construct
  source 1..23: mol_type = other DNA
tagcttttttggacagtacggat

SEQ ID NO: 151 - DNA, 22 bp, synthetic construct
  source 1..22: mol_type = other DNA
ttcgagctctcagacgagaagg

SEQ ID NO: 152 - DNA, 22 bp, synthetic construct
  source 1..22: mol_type = other DNA
ccacattgcggttttgaccaat

SEQ ID NO: 153 - DNA, 25 bp, synthetic construct
  source 1..25: mol_type = other DNA
agtgtatttgaccaacaccaccata

SEQ ID NO: 154 - DNA, 22 bp, synthetic construct
  source 1..22: mol_type = other DNA
cccgttatacaaacatgcagcc

SEQ ID NO: 155 - DNA, 22 bp, synthetic construct
  source 1..22: mol_type = other DNA
gtgttcctttccatctggggac

SEQ ID NO: 156 - DNA, 25 bp, synthetic construct
  source 1..25: mol_type = other DNA
gtttttaattaatgcactcaaatgc

SEQ ID NO: 157 - DNA, 22 bp, synthetic construct
  source 1..22: mol_type = other DNA
gaagtgctcagcatgtcgaaga

SEQ ID NO: 158 - DNA, 22 bp, synthetic construct
  source 1..22: mol_type = other DNA
ggacctatcgcctctgtcaact

SEQ ID NO: 159 - DNA, 22 bp, synthetic construct
  source 1..22: mol_type = other DNA
tcaaagtcgttgaaggctggtc

SEQ ID NO: 160 - DNA, 22 bp, synthetic construct
  source 1..22: mol_type = other DNA
atcttcaagtctctgcgcgatc

SEQ ID NO: 161 - DNA, 22 bp, synthetic construct
  source 1..22: mol_type = other DNA
ggtattgtttccttaattgtcg

SEQ ID NO: 162 - DNA, 22 bp, synthetic construct
  source 1..22: mol_type = other DNA
tgagagctatttctttggcccc

SEQ ID NO: 163 - DNA, 23 bp, synthetic construct
  source 1..23: mol_type = other DNA
tggagacccaaataacatggaca

SEQ ID NO: 164 - DNA, 22 bp, synthetic construct
  source 1..22: mol_type = other DNA
actaggatgagtcccaatggct

SEQ ID NO: 165 - DNA, 25 bp, synthetic construct
  source 1..25: mol_type = other DNA
gagtttgtttctataattgcgaaag

SEQ ID NO: 166 - DNA, 22 bp, synthetic construct
  source 1..22: mol_type = other DNA
tgctgacaaaatgactgtcgtc

SEQ ID NO: 167 - DNA, 22 bp, synthetic construct
  source 1..22: mol_type = other DNA
acggaaggtgtacctgagtcta

SEQ ID NO: 168 - DNA, 24 bp, synthetic construct
  source 1..24: mol_type = other DNA
agtcatgtcagttatgtatcgcga

SEQ ID NO: 169 - DNA, 25 bp, synthetic construct
  source 1..25: mol_type = other DNA
gtagttttttactccaattctatgc

SEQ ID NO: 170 - DNA, 28 bp, synthetic construct
  source 1..28: mol_type = other DNA
tgtagatttttagagactcgaactgtgt

SEQ ID NO: 171 - DNA, 22 bp, synthetic construct
  source 1..22: mol_type = other DNA
attgctaagggctttcaccgaa

SEQ ID NO: 172 - DNA, 27 bp, synthetic construct
  source 1..27: mol_type = other DNA
gtgtgttttttatcattaaataagctg

SEQ ID NO: 173 - DNA, 17 bp, synthetic construct
  source 1..17: mol_type = other DNA
acccaactgaatggagc

SEQ ID NO: 174 - DNA, 19 bp, synthetic construct
  source 1..19: mol_type = other DNA
acgcacttgacttgtcttc

(19) United States
(12) Patent Application Publication
(10) Pub. No.: US 2025/0122586 A1
(43) Pub. Date: Apr. 17, 2025

(54) COMPOSITIONS AND METHODS FOR THE DETECTION OF H3N2 INFLUENZA VARIANTS

(71) Applicant: THE TRANSLATIONAL GENOMICS RESEARCH INSTITUTE, Phoenix, AZ (US)

(72) Inventors: Crystal Hepp, Flagstaff, AZ (US); Daryn Erickson, Flagstaff, AZ (US)

(21) Appl. No.: 18/919,391

(22) Filed: Oct. 17, 2024

Related U.S. Application Data
(60) Provisional application No. 63/591,100, filed on Oct. 17, 2023.

(51) Int. Cl. C12Q 1/70; A61K 31/196; A61K 31/215; A61K 31/351; A61K 31/5383

(52) U.S. Cl. CPC C12Q 1/701; A61K 31/196; A61K 31/215; A61K 31/351; A61K 31/5383; C12Q 2600/156

(57) ABSTRACT

A method of detecting H3N2 subtype of the influenza A virus is provided. The method may include producing an amplicon by amplifying a nucleic acid segment from a sample obtained from the subject with, at least one forward primer selected from the group consisting of SEQ ID NOS: 1-43, and at least one reverse primer selected from the group consisting of SEQ ID NOS: 44-86, and sequencing the amplicon to detect the presence of the H3N2 influenza virus variant in the sample.

### RELATED APPLICATIONS

This application claims the benefit of U.S. Provisional Patent Application No. 63/591,100, filed Oct. 17, 2023, to The Translational Genomics Research Institute, titled “COMPOSITIONS AND METHODS FOR THE DETECTION OF H3N2 INFLUENZA VARIANTS,” the entirety of the disclosure of which is hereby incorporated by this reference.

### INCORPORATION-BY-REFERENCE OF MATERIAL ELECTRONICALLY FILED

The official copy of the sequence listing is submitted electronically in ST.26 XML format having the file name “91482-269US-PAT_SeqList.xml” created on Oct. 17, 2024, and having a size of 156,463 bytes, and is filed concurrently with the specification. The Sequence Listing ST.26 XML file is part of the specification and is herein incorporated by reference in its entirety.

### TECHNICAL FIELD

The subject matter disclosed herein is generally directed to detection of influenza virus variants, in particular, influenza A subtype H3N2.

### BACKGROUND

Presently, H1N1 and H3N2 are the most frequently detected subtypes of Influenza A. Influenza A virus subtype H3N2 (A/H3N2) is a subtype of influenza A virus. All subtypes of influenza A virus share a negative-sense, segmented RNA genome. Some human-adapted strains of A/H3N2 are endemic in humans and are one cause of seasonal influenza (flu). Subtypes of influenza A virus are defined by the combination of the antigenic H and N proteins in the viral envelope; for example, “H3N2” designates an influenza A virus subtype that has a type-3 hemagglutinin (H) protein and a type-2 neuraminidase (N) protein.

Each year, three influenza strains are chosen for inclusion in the forthcoming year's seasonal flu vaccination by the Global Influenza Surveillance and Response System of the World Health Organization (WHO). Since 1999, every annual formulation of seasonal flu vaccination has included one strain of A/H3N2. Thus, there is a need for improved methods of detection of H3N2 influenza.

Citation or identification of any document in this application is not an admission that such a document is available as prior art to the present invention.

### SUMMARY

The present invention is directed to a method of detecting an influenza virus variant, including the H3N2 subtype of Influenza A, within a sample from a subject.

In one aspect, the present invention provides for a method of detecting an H3N2 influenza virus in a sample, comprising: producing one or more amplicons by amplifying one or more nucleic acid segments from the sample using, (a) at least one forward primer selected from the group consisting of SEQ ID NOS: 1-43, and (b) at least one reverse primer selected from the group consisting of SEQ ID NOS: 44-86; and sequencing the one or more amplicons to detect the presence of H3N2 influenza virus in the sample.

In certain embodiments, the biological sample is an environmental sample. In certain embodiments, the sample is from a subject. In certain embodiments, the sample is obtained from an animal subject. In certain embodiments, the sample is obtained from a human subject.

In certain embodiments, the sequencing is next-generation sequencing (NGS). In certain embodiments, H3N2 influenza virus is detected in the sample if the amplicon sequences are greater than or equal to 90% identical to an H3N2 influenza virus reference sequence. In certain embodiments, H3N2 influenza virus is detected in the sample if the amplicon sequences are greater than or equal to 95% identical to an H3N2 influenza virus reference sequence. In certain embodiments, H3N2 influenza virus is detected in the sample if the amplicon sequences include 10 or less single nucleotide polymorphisms (SNPs) as compared to an H3N2 influenza virus reference sequence.

In certain embodiments, if H3N2 influenza virus is detected in the sample the subject is treated with an antiviral treatment. In certain embodiments, the antiviral treatment is selected from the group consisting of oseltamivir phosphate, zanamivir, peramivir, and baloxavir marboxil.

In another aspect, the present invention provides for a method of detecting H3N2 in a sample, comprising: performing real-time PCR to detect nucleic acid segments from the sample with, (a) at least one forward primer selected from the group consisting of SEQ ID NOS: 87-129, and (b) at least one reverse primer selected from the group consisting of SEQ ID NOS: 130-172; and detecting the presence of H3N2 in the sample by comparing the amplification to a control value. In certain embodiments, H3N2 influenza virus is detected in the sample if the amplification reaches a threshold value at an earlier cycle than background amplification or a negative control value. In certain embodiments, amplification is detected by fluorescence of a detectably labeled probe that is specific to a sequence within an amplicon obtained by amplification with the forward and reverse primer during real-time PCR.

In certain embodiments, the sample is an environmental sample. In certain embodiments, the sample is from a subject. In certain embodiments, the sample is obtained from an animal subject. In certain embodiments, the sample is obtained from a human subject.

In certain embodiments, if H3N2 influenza virus is detected in the sample the subject is treated with an antiviral treatment. In certain embodiments, the antiviral treatment is selected from the group consisting of oseltamivir phosphate, zanamivir, peramivir, and baloxavir marboxil.

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

The foregoing and other aspects, features, and advantages will be apparent from the DESCRIPTION and DRAWINGS, and from the CLAIMS if any are included.

### BRIEF DESCRIPTION OF THE DRAWINGS

An understanding of the features and advantages of the present invention will hereinafter be described in conjunction with the appended and/or included DRAWINGS, where like designations denote like elements, and:

FIG. 1—illustrates DNA bait-capture results.

FIG. 2—illustrates a H3N2 read pile-up with 100% breadth of coverage.

FIG. 3A-FIG. 3B—illustrates tiled amplicon sequencing approaches. FIG. 3A. A tiled amplicon sequencing experiment. FIG. 3B. A tiled amplicon sequencing experiment showing 99.5% breadth of coverage.

FIG. 4—illustrates a phylogenomic analysis.

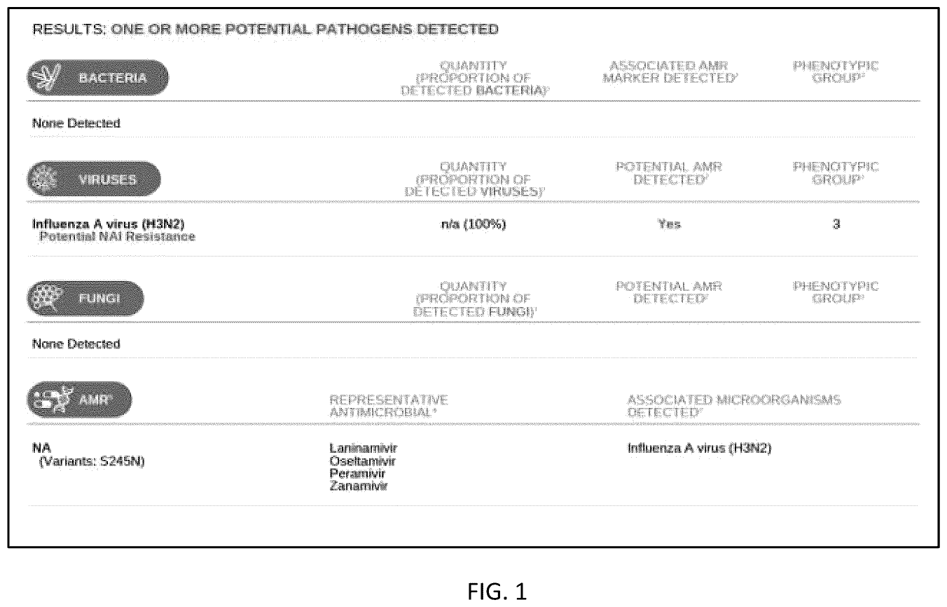

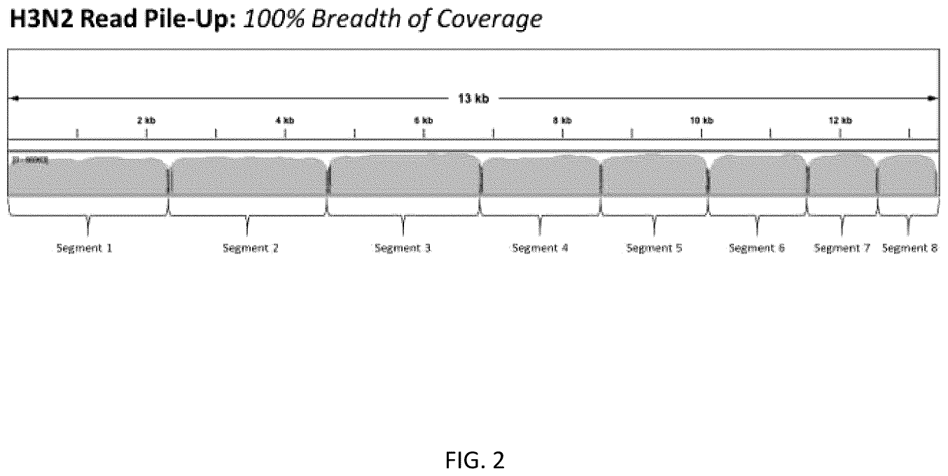

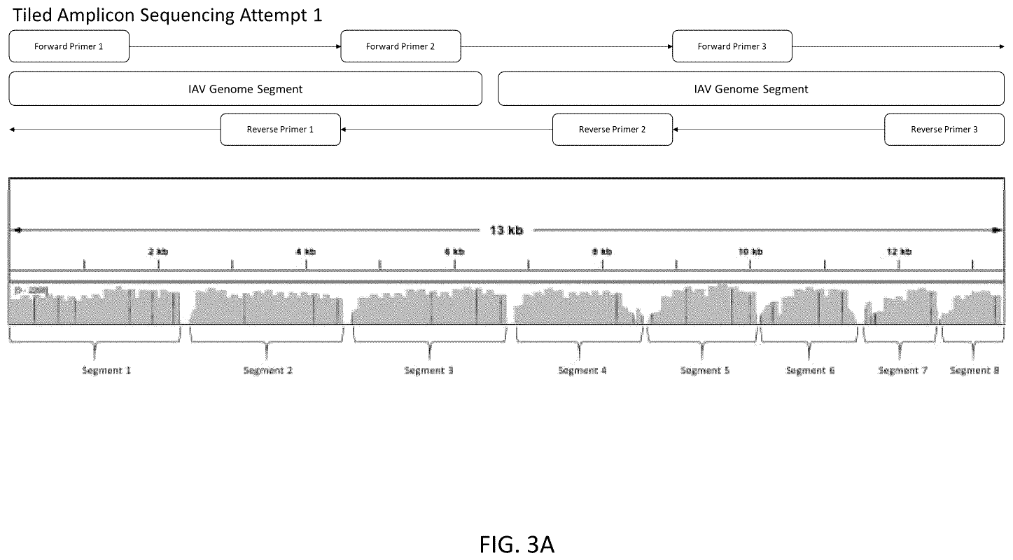

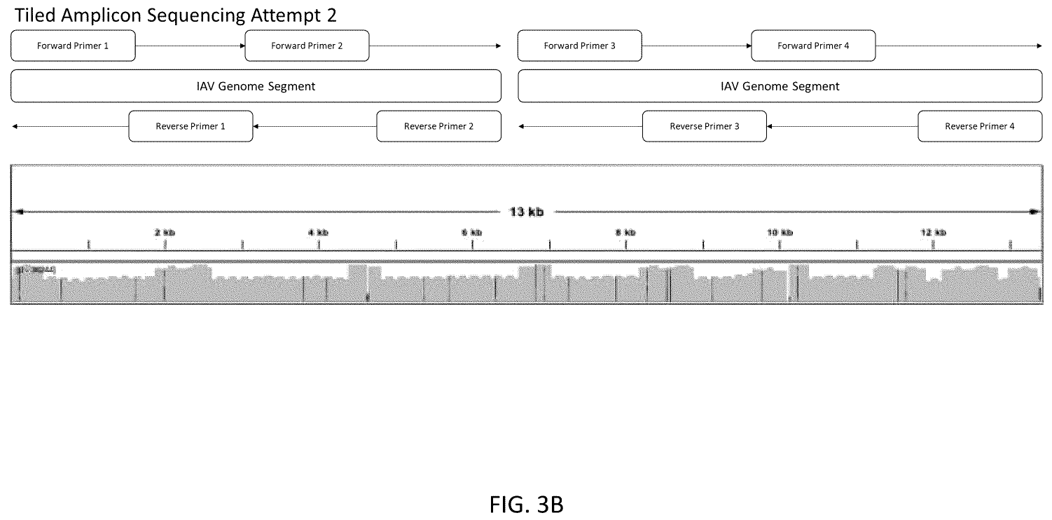

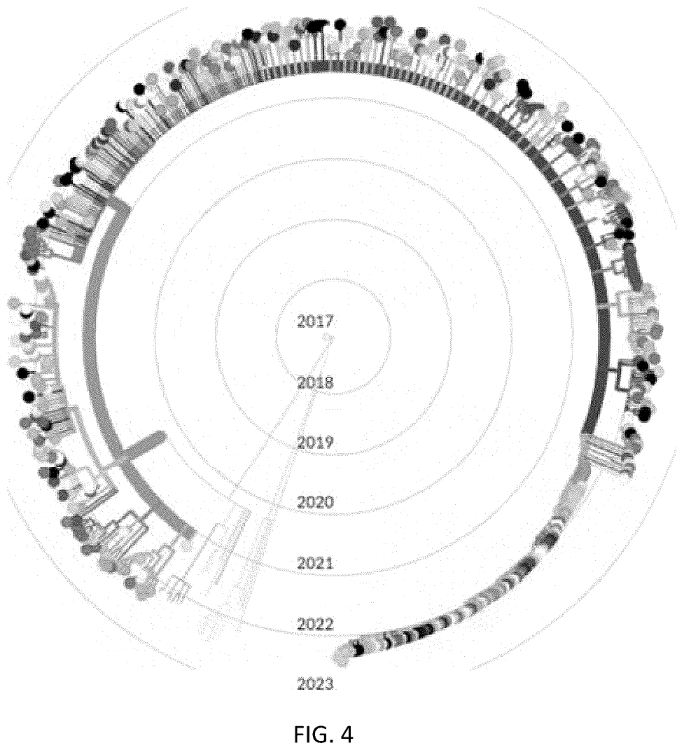

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The figures herein are for illustrative purposes only and are not necessarily drawn to scale.

### DETAILED DESCRIPTION OF THE EXAMPLE EMBODIMENTS

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. Thus, for example, reference to “a step” includes reference to one or more of such steps. Reference to a particular numerical value includes at least that particular value, unless the context clearly dictates otherwise.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

The term “plurality”, as used herein, means more than one.

As used herein, the term “comprise,” and conjugations or any other variation thereof, are used in its non-limiting sense to mean that items following the word are included, but items not specifically mentioned are not excluded. Throughout the description and claims of this specification, the words “comprise” and “contain” and variations of the words, for example “comprising” and “comprises”, mean “including but not limited to”, and are not intended to (and do not) exclude other components.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination. For example, the foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods of detecting an influenza virus variant within a sample from a subject, including detecting the H3N2 subtype of Influenza A. Presently, H1N1 and H3N2 are the most frequently detected subtypes of Influenza A, but given precautions taken during the global coronavirus pandemic, the present study was interested in determining if different subtypes of Influenza A would arise. Additionally, it was hypothesized that precautions taken during the SARS-COV-2 omicron variant surge may have substantially limited influenza introductions over a short period of time within a Northern Arizona community.

Residual rapid test swabs were collected from samples that were confirmed to be positive for Influenza A from the Northern Arizona community. The swabs were evaluated for viral load of Influenza A with Reverse Transcription-qPCR (RT-qPCR). To determine which subtypes were circulating in the community, multiple samples with high viral loads were selected for whole-genome sequencing with a DNA bait capture kit. This approach allowed for enrichment of full Influenza A genomes from three of the residual rapid test samples, all of which aligned to the H3N2 subtype of Influenza A. To feasibly sequence and characterize H3N2 genomes from the remaining samples, a tiled amplicon primer scheme was designed based on the DNA bait-capture output to span the entirety of contemporary H3N2 genomes. While tiled amplicon sequencing is used to enrich small portions of large pathogen genomes and entire unsegmented viral genomes, it has not been previously used to amplify whole segmented viral genomes.

Application of the primer scheme resulted in near complete H3N2 genomes from the remaining nasal swabs. However, it failed to amplify an H3N2 positive control from a sample collected in 2012, indicating the necessity of closely related references for development of tiled amplicon sequencing schemes. A phylogenetic tree revealed that at least four introductions occurred within the sampled population, and only one of these clades indicated a rapid outbreak over a short time-frame. This study demonstrates the utility of DNA baits to capture distantly related viruses that can be leveraged for more feasible amplicon approaches. For example, primers can be generated specific for amplifying overlapping amplicons from virus genomes present in the population, such that sequencing the amplicons can provide the whole genome sequences of the viruses. Applicants provide primer sequences specific to a population of H3N2 influenza A.

### Terminology and Definitions

In describing the embodiments herein, the following terminology will be used in accordance with the definitions and explanations set out below. Notwithstanding, other terminology, definitions, and explanations may be found throughout this document as well.

As used herein, “amplification reaction” refers to a method of detecting target nucleic acid by in vitro amplification of DNA or RNA.

As used herein, “polymerase chain reaction (PCR)” refers to the amplification of a specific DNA sequence, termed target or template sequence, that is present in a mixture, by adding two or more short oligonucleotides, also called primers, that are specific for the terminal or outer limits of the template sequence. The template-primers mixture is subjected to repeated cycles of heating to separate (melt) the double-stranded DNA and cooling in the presence of nucleotides and DNA polymerase such that the template sequence is copied at each cycle.

A real-time polymerase chain reaction (real-time PCR, or qPCR when used quantitatively) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively and semi-quantitatively (i.e., above/below a certain amount of DNA molecules). A real-time polymerase chain reaction (real-time PCR, or qPCR when used quantitatively) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively and semi-quantitatively (i.e., above/below a certain amount of DNA molecules). A commonly employed method of DNA quantification by real-time PCR relies on plotting fluorescence against the number of cycles on a logarithmic scale. A threshold for detection of DNA-based fluorescence is set 3-5 times of the standard deviation of the signal noise above background. The number of cycles at which the fluorescence exceeds the threshold is called the threshold cycle (Ct).

The term “primer” refers to DNA oligonucleotides complementary to a region of DNA and serves as the initiation of amplification reaction from the 5′ to 3′ direction.

The term “primer pair” refers to the forward and reverse primers in an amplification reaction leading to amplification of a double-stranded DNA region of the target.

The term “target” refers to a nucleic acid region bound by a primer pair that is amplified through an amplification reaction. The PCR “product” or “amplicon” is the amplified nucleic acid resulting from PCR of a set of primer pairs.

The term “multiplex amplification reaction” herein refers to the detection of more than one template in a mixture by the addition of more than one set of oligonucleotide primers (e.g., more than one set of primer pairs).

“Amplification” is a special case of nucleic acid replication involving template specificity. Amplification may be a template-specific replication or a non-template-specific replication (i.e., replication may be specific template-dependent or not). Template specificity is here distinguished from fidelity of replication (synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out. The amplification process may result in the production of one or more amplicons.

The term “template” refers to nucleic acid originating from a sample that is analyzed for the presence of one or more markers. In contrast, “background template” or “control” is used in reference to nucleic acid other than sample template that may or may not be present in a sample. Background template is most often inadvertent. It may be the result of carryover, or it may be due to the presence of nucleic acid contaminants sought to be purified out of the sample. For example, nucleic acids from organisms other than those to be detected may be present as background in a test sample.

In addition to primers and probes, template specificity is also achieved in some amplification techniques by the choice of enzyme. Amplification enzymes are enzymes that, under the conditions in which they are used, will process only specific sequences of nucleic acid in a heterogeneous mixture of nucleic acid. Other nucleic acid sequences will not be replicated by this amplification enzyme. Similarly, in the case of T7 RNA polymerase, this amplification enzyme has a stringent specificity for its own promoters (Chamberlin et al. (1970) Nature (228):227). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics (4):560). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.) (1989) PCR Technology, Stockton Press).

The term “amplifiable nucleic acid” refers to nucleic acids that may be amplified by any amplification method. It is contemplated that “amplifiable nucleic acid” will usually comprise “sample template.” The terms “PCR product,” “PCR fragment,” “amplification product,” and “amplicon” refer to the resultant mixture of compounds after two or more cycles of the PCR steps of denaturation, annealing and extension. These terms encompass the case where there has been amplification of one or more segments of one or more target sequences.

The present invention encompasses embodiments wherein a sample is a biological sample. As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The present invention encompasses embodiments wherein a sample is an environmental sample. Non-limiting examples of an environmental sample include wastewater, air, water from aircraft lavatories, water from schools, and environmental samples in poultry areas.

Any reference to a gene is a reference made to the gene and the gene product (e.g., protein).

### Methods of Detecting H3N2 Subtype of the Influenza A Virus

In example embodiments, the present invention comprises a method of detecting H3N2 subtypes of the influenza A virus comprising the steps of producing an amplicon by amplifying one or more nucleic acid segments from a sample obtained from a subject or from an environmental sample with, at least one forward primer selected from the group consisting of SEQ ID NOS: 1-43, and at least one reverse primer selected from the group consisting of SEQ ID NOS: 44-86, and sequencing the amplicon to detect the H3N2 influenza virus variant. In example embodiments, one or more primer pairs consisting of a forward primer and a reverse primer that hybridize to the ends of a target amplicon are used to amplify one or more amplicons, which can then be sequenced. In example embodiments, primer pairs are selected to amplify overlapping amplicons to obtain amplicons that cover an entire region of a target sequence (i.e., a tiled amplicon sequencing scheme).

In example embodiments, the primers generate amplicons that allows for the complete overlap of forward and reverse paired-end sequencing reads from the same DNA molecule (see, e.g., Colman R E, Schupp J M, Hicks N D, et al. Detection of Low-Level Mixed-Population Drug Resistance in Mycobacterium tuberculosis Using High Fidelity Amplicon Sequencing. PLOS One. 2015; 10 (5): e0126626). This provides two independent base calls at each position within the same DNA fragment, which significantly lowers the probability of an erroneous base call by orders of magnitude. For example, forward primers SEQ ID NOS: 1-43 and reverse primers SEQ ID NOS: 44-86 include universal tail sequences.

Detection according to some embodiments of the disclosure may comprise contacting the amplified nucleic acid with a probe; and detecting the hybridization of probe with the amplified nucleic acid. Detection may be performed by a variety of methods, such as but not limited to, by a nucleic acid amplification reaction. In some embodiments the amplification reaction maybe an end-point determination or the amplification reaction maybe quantitative. The quantification may be a real-time PCR method. In some embodiments, the real-time PCR may be a SYBR Green Assay or a TAQMAN Assay. Detection, in various embodiments, maybe performed by hybridization using probes specific to target sequences. According to various embodiments, combinations of amplification and hybridization may be used for detection.

As used herein, “real-time PCR” may refer to the detection and quantitation of a DNA or a surrogate thereof in a sample. In some embodiments, the amplified segment or “amplicon” can be detected in real time using a 5′-nuclease assay, particularly the TaqMan® assay as described by e.g., Holland et al. (Proc. Natl. Acad. Sci. USA 88:7276-7280, 1991); and Heid et al. (Genome Research 6:986-994, 1996). For use herein, a TaqMan® nucleotide sequence to which a TaqMan® probe binds can be designed into the primer portion, or known to be present in DNA of a sample. TaqMan® probe refer to probes containing a donor fluorescent moiety at the 5′-end and an acceptor fluorescent moiety at the 3′-end that quenches the fluorescence emitted from the donor molecule due to their close proximity. In some embodiments, the PCR methods use end-point PCR and a positive result is obtained when there is a detectable signal after the PCR is finished. Real-time and end-point PCR methods useful in accordance with the present methods and compositions include, but are not limited to, fluorescence resonance energy transfer (FRET), TAQMAN®, Molecular Beacons, Amplifluor®, Scorpion™, Plexor™, BHQplus™.

Detection method embodiments using a TaqMan® probe sequence comprise combining the test sample with PCR reagents, including a primer set having a forward primer and a reverse primer, a DNA polymerase, and a fluorescent detector oligonucleotide TaqMan® probe, as well as dNTP's and a salt, to form an amplification reaction mixture; subjecting the amplification reaction mixture to successive cycles of amplification to generate a fluorescent signal from the detector probe; and quantitating the nucleic acid presence based on the fluorescent signal cycle threshold of the amplification reaction.

In example embodiments, the presence and quantity of H3N2 subtypes of the influenza A virus subtypes can be detected by real time PCR using at least one forward primer selected from the group consisting of SEQ ID NOS: 87-129, and at least one reverse primer selected from the group consisting of SEQ ID NOS: 130-172. In example embodiments, amplification is detected using fluorescent probes specific to a sequence between the forward and reverse primers.

As described in greater detail herein, some embodiments of the invention may include amplicon-based sequencing of the one or more markers to make the aforementioned determinations. Some embodiments of the invention include systems and methods of preparing samples for one or more downstream processes that can be used for assessing one or more markers for any of the previously mentioned purposes. Some embodiments of the invention may comprise a universal indexing sequencing strategy for use in downstream sequencing platform processes. By way of example only, some embodiments of the invention comprise a universal indexing sequencing strategy that can be used to amplify multiple genomic regions (e.g., markers, as described below) from a DNA sample simultaneously in a single reaction for the sequencing of one or more amplicons. One or more embodiments of the invention can be used with any desired sequencing platform, such as the ILLUMINA® Next Generation Sequencing (e.g., MiSEQ) platform, Life Technologies' Ion Torrent System, or any other sequencing system now known or developed in the future.

In example embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56 (2):61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi: 10.1002/0471142727.mb0422s107 (2014). PMCID: 4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437:376-80); Schneider and Dekker (Nat Biotechnol. 2012 Apr. 10; 30 (4):326-8); Ronaghi et al (Analytical Biochemistry 1996 242:84-9); Shendure et al (Science 2005 309:1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

Some embodiments may be configured to enable relatively simple, rapid (e.g., microorganism-culture independent), inexpensive, and efficient preparation of samples for use on, in, and/or with downstream sequencing platforms. For example, some embodiments may use a sequence coupled to one or more oligonucleotides/primers (as used herein, oligonucleotides and primers are used interchangeably). More specifically, one or more amplicons per sample can be generated using a hybrid oligonucleotide that is designed for amplification of a marker and incorporation of at least one universal tail sequence into the resulting amplicon. As a result, additional steps that may be conventionally required to prepare samples for sequencing can be limited or removed entirely. Further information regarding the universal tail, amplicon-based sequencing strategy can be found in PCT/US2014/064890, which is hereby incorporated by reference in its entirety for all purposes.

In some embodiments, the methodology may include performing downstream sequencing on one or more amplicons. For example, in order to minimize and/or eliminate the need for cultures of microorganisms and/or viruses or large inputs of nucleic acids, methodologies of the instant invention may include an initial PCR step to create amplicons that correspond to the one or more pre-selected markers. As such, some embodiments require only limited amounts of starting material are necessary and the starting material need not be of high quality (e.g., genomic DNA, crude DNA extracts, single stranded DNA, RNA, cDNA, etc.). In contrast, many conventional sample preparation systems may require relatively large amounts of starting material of relatively high quality, which can limit the use of some conventional systems.

Some embodiments of the invention can be used for and/or in complement with high-throughput amplicon sequencing of markers, which can be very useful for a variety of molecular genetic genotyping/predicted-phenotyping applications, including clinical sample analysis. For example, use of the systems and methods of the invention can be employed with sequencing platforms to provide rapid, high-yield sequence data, which can enable the sequencing of multiple markers/amplicons from many samples in a relatively short period of time. Specifically, in some embodiments, amplicons can be selected and PCR reactions can be designed to provide information that can be used to make clinically relevant determinations after sequencing of the amplicons.

In some preferred aspects, the methodology may include creating a series of oligonucleotides designed to provide multiplexed amplification of one or more markers to produce the desired amplicons. In particular, the one or more markers and amplicons thereof can be selected/amplified to provide users with clinically relevant information related to identification of one or more potentially infectious microorganisms and/or viruses and phenotypic and genotypic information about the microorganisms and/or viruses. After production of the amplicons (e.g., via PCR amplification), which may include the universal tail sequences, the method may include processing the resulting amplicons for downstream sequencing and thereafter sequencing the processed amplicons. After processing and analysis of the resulting sequencing data, one of skill in the art can make any necessary determinations regarding the identification of one or more microorganisms and/or viruses that may have been contained within the sample and predicted-phenotypic and/or genotypic information revealed.

Generally, some embodiments of the present invention can be used to detect, identify, assess, sequence, or otherwise evaluate a marker. A marker may be any molecular structure produced by a cell, expressed inside the cell, accessible on the cell surface or secreted by the cell. A marker may be any protein, carbohydrate, fatty acid, nucleic acid, catalytic site, or any combination of these such as an enzyme, glycoprotein, cell membrane, virus, a particular cell, or other uni- or multimolecular structure. A marker may be represented by a sequence of a nucleic acid or any other molecules derived from the nucleic acid. Examples of such nucleic acids include viral genome (e.g., viral RNA genome), miRNA, tRNA, siRNA, mRNA, cDNA, genomic DNA sequences, single-stranded DNA, or complementary sequences thereof. Alternatively, a marker may be represented by a protein sequence. The concept of a marker is not limited to the exact nucleic acid sequence or protein sequence or products thereof; rather it encompasses all molecules that may be detected by a method of assessing the marker. Without being limited by the theory, the detection, identification, assessment, sequencing, or any other evaluation of the marker may encompass an assessment of a change in copy number (e.g., copy number of a gene or other forms of nucleic acid) or in the detection of one or more translocations. Moreover, in some embodiments, the marker may be relevant to a particular phenotype or genotype. By way of example only, in some embodiments, the marker may be related to phenotypes including antibiotic resistance, virulence, or any other phenotype.

Therefore, examples of molecules encompassed by a marker represented by a particular sequence further include alleles of the gene used as a marker. An allele includes any form of a particular nucleic acid that may be recognized as a form of the particular nucleic acid on account of its location, sequence, or any other characteristic that may identify it as being a form of the particular gene. Alleles include but need not be limited to forms of a gene that include point mutations, silent mutations, deletions, frameshift mutations, single nucleotide polymorphisms (SNPs), inversions, translocations, heterochromatic insertions, and differentially methylated sequences relative to a reference gene, whether alone or in combination. An allele of a gene may or may not produce a functional protein; may produce a protein with altered function, localization, stability, dimerization, or protein-protein interaction; may have overexpression, underexpression or no expression; may have altered temporal or spatial expression specificity; or may have altered copy number (e.g., greater or less numbers of copies of the allele). An allele may also be called a mutation or a mutant. An allele may be compared to another allele that may be termed a wild type form of an allele. In some cases, the wild type allele is more common than the mutant. A SNP is a substitution of a single nucleotide that occurs at a specific position in the genome, where each variation is present to some appreciable degree within a population (e.g. >1%).

In some aspects, the markers may include one or more sets of amplifiable nucleic acids that can provide diagnostic information about the microorganisms and/or viruses. For example, the markers may include amplifiable nucleic acid sequences that can be used to assess the presence and/or absence of one or more microorganism and/or virus that may have the potential to cause a diseased state in the subject. In some embodiments, the markers may include amplifiable nucleic acid sequences that can be used to identify one or more of the following exemplary microorganisms and/or viruses: Influenza A, H3N2 subtype.

In some embodiments, the methods may include the use of one or more than one marker per microorganism and/or virus. Moreover, in some embodiments, one or more of the microorganisms and/or viruses may not be considered pathogenic to certain subjects, but the methodology employed herein can still rely on detection of pathogenic and non-pathogenic microorganisms and/or viruses for differential diagnoses/diagnostics. In some embodiments, the oligonucleotides (with or without the universal tail sequences detailed herein) listed in Table 1 and Table 2 can be used with embodiments of the invention to amplify one or more markers from the microorganisms and/or viruses to provide diagnostic/identification information to the user.

Moreover, in some embodiments, one or more the markers associated with the plurality of microorganisms and/or viruses can be amplified in a multiplex manner. For example, in some aspects, nucleic acids can be obtained from the sample and the oligonucleotides used to amplify one or more of the markers used to identify/diagnose can be added to a single mixture to produce a plurality of amplicons in a single reaction mixture. In other aspects, the oligonucleotides can be added to multiple mixtures to provide for the creation of multiple amplicons in multiple mixtures.

Moreover, in some embodiments, one or more the markers can be amplified in a multiplex manner. For example, in some aspects, nucleic acids can be obtained from the sample and the oligonucleotides used to amplify one or more of the markers used to identify the strain of the microorganism and/or virus can be added to a single mixture to produce a plurality of amplicons in a single reaction mixture. In other aspects, the oligonucleotides can be added to multiple mixtures to provide for the creation of multiple amplicons in multiple mixtures. In some aspects, amplification of the markers used to identify microorganisms and/or viruses/diagnose an infection can also occur in a multiplex manner such that some or all of the amplicons are generated in a single reaction for a particular sample. In other aspects, amplification of the markers used to identify microorganisms and/or viruses/diagnose an infection can occur in multiple reaction vessels. Overall, as described in greater detail below, regardless of the multiplex nature of some embodiments of the invention, after amplification of the markers, the method may include processing and sequencing the resulting amplicons to provide information related to the identification, characterization, and strain identity of one or more microorganisms and/or viruses that may be present within the sample.

Some embodiments of the invention may comprise the use of one or more methods of amplifying a nucleic acid-based starting material (i.e., a template, including genomic DNA, crude DNA extract, single-stranded DNA, double-stranded DNA, cDNA, RNA, or any other single-stranded or double-stranded nucleic acids). Nucleic acids may be selectively and specifically amplified from a template nucleic acid contained in a sample. In some nucleic acid amplification methods, the copies are generated exponentially. Examples of nucleic acid amplification methods known in the art include: polymerase chain reaction (PCR), ligase chain reaction (LCR), self-sustained sequence replication (3SR), nucleic acid sequence based amplification (NASBA), strand displacement amplification (SDA), amplification with Qβ replicase, whole genome amplification with enzymes such as φ29, whole genome PCR, in vitro transcription with T7 RNA polymerase or any other RNA polymerase, or any other method by which copies of a desired sequence are generated.

In addition to genomic DNA, any polynucleotide sequence can be amplified with an appropriate set of primer molecules. In particular, the amplified segments created by the PCR process itself are, themselves, efficient templates for subsequent PCR amplifications.

PCR generally involves the mixing of a nucleic acid sample, two or more primers or oligonucleotides (primers and oligonucleotides are used interchangeably herein) that are designed to recognize the template DNA, a DNA polymerase, which may be a thermostable DNA polymerase such as Taq or Pfu, and deoxyribose nucleoside triphosphates (dNTP's). In some embodiments, the DNA polymerase used can comprise a high fidelity Taq polymerase such that the error rate of incorrect incorporation of dNTPs is less than one per 1,000 base pairs. Reverse transcription PCR, quantitative reverse transcription PCR, and quantitative real time reverse transcription PCR are other specific examples of PCR. In general, the reaction mixture is subjected to temperature cycles comprising a denaturation stage (typically 80-100° C.), an annealing stage with a temperature that is selected based on the melting temperature (Tm) of the primers and the degeneracy of the primers, and an extension stage (for example 40-75° C.). In real-time PCR (RT-PCR) analysis, additional reagents, methods, optical detection systems, and devices known in the art are used that allow a measurement of the magnitude of fluorescence in proportion to concentration of amplified template. In such analyses, incorporation of fluorescent dye into the amplified strands may be detected or measured.

Either primers or primers along with probes allow a quantification of the amount of specific template DNA present in the initial sample. In addition, RNA may be detected by PCR analysis by first creating a DNA template from RNA through a reverse transcriptase enzyme (i.e., the creation of cDNA). The marker expression may be detected by quantitative PCR analysis facilitating genotyping analysis of the samples.

In some forms of PCR assays, quantification of a target in an unknown sample is often required. Such quantification may be determined in reference to the quantity of a control sample. The control sample starting material/template may be co-amplified in the same tube in a multiplex assay or may be amplified in a separate tube. Generally, the control sample contains template at a known concentration. The control sample template may be a plasmid construct comprising only one copy of the amplification region to be used as quantification reference. To calculate the quantity of a target in an unknown sample, various mathematical models are established. Calculations are based on the comparison of the distinct cycle determined by various methods, e.g., crossing points (CP) and cycle threshold values (Ct) at a constant level of fluorescence; or CP acquisition according to established mathematic algorithm.

Some embodiments of the invention may comprise a multiplex assay. As used herein, the term “multiplex” refers to the production of more than one amplicon, PCR product, PCR fragment, amplification product, etc. in a single reaction vessel. In other words, multiplex is to be construed as the amplification of more than one marker-specific sequences within a PCR reaction or assay within the same PCR assay mixture (e.g., more than one amplicon is produced within a single vessel that contains all of the reagents necessary to perform a PCR reaction). In some embodiments, a step prior to performing the PCR (or RT-PCR, quantitative RT-PCR, etc.) reaction can occur such that sets of primers and/or primers and probes are designed, produced, and optimized within a given set of reaction conditions to ensure proper amplicon production during the performance of the PCR.

The algorithm for Ct values in real time-PCR calculates the cycle at which each PCR amplification reaches a significant threshold. The calculated Ct value is proportional to the number of marker copies present in the sample, and the Ct value is a precise quantitative measurement of the copies of the marker found in any sample. In other words, Ct values represent the presence of respective marker that the primer sets are designed to recognize. If the marker is missing in a sample, there should be no amplification in the Real Time-PCR reaction.

Alternatively, the Cp value may be utilized. A Cp value represents the cycle at which the increase of fluorescence is highest and where the logarithmic phase of a PCR begins. The LIGHTCYCLER® 480 Software calculates the second derivatives of entire amplification curves and determines where this value is at its maximum. By using the second-derivative algorithm, data obtained are more reliable and reproducible, even if fluorescence is relatively low.

The various and non-limiting embodiments of the PCR-based method detecting marker expression level as described herein may comprise one or more probes and/or primers. Generally, the probe or primer contains a sequence complementary to a sequence specific to a region of the nucleic acid of the marker gene. A sequence having less than 60% 70%, 80%, 90%, 95%, 99% or 100% identity to the identified gene sequence may also be used for probe or primer design if it is capable of binding to its complementary sequence of the desired target sequence in marker nucleic acid.

Some embodiments of the invention may include a method of comparing a marker in a sample relative to one or more control samples. A control may be any sample with a previously determined level of expression. A control may comprise material within the sample or material from sources other than the sample. Alternatively, the expression of a marker in a sample may be compared to a control that has a level of expression predetermined to signal or not signal a cellular or physiological characteristic. This level of expression may be derived from a single source of material including the sample itself or from a set of sources.

In example embodiments, detection of amplification (e.g., Ct, Cp value) in a real-time PCR assay for a sample obtained from a subject is compared to a control value. In example embodiments, the control value is the amplification determined or obtained for other samples. In example embodiments, the samples used for the control value are positive for the influenza subtype. In example embodiments, the control value is the average amplification value for a set of samples positive for the influenza subtype. In example embodiments, the control value is obtained from a set of samples obtained from subjects negative for the influenza subtype.

In example embodiments, the control value is background amplification. As used herein “background amplification” refers to the amplification by a forward and reverse PCR primer detected in a sample not containing nucleic acids or in a sample containing nucleic acids and not containing nucleic acid segments that the primers specifically amplify. In example embodiments, a fluorescent probe specific for an amplicon produced by a forward and reverse primer is used to detect amplification. Any amplification detected in a sample not containing a target nucleic acid segment is background amplification. In example embodiments, during real time PCR, background amplification can occur at later cycles (e.g., after cycle 34, 35, 36, 37, 38, 39, or 40).

In some embodiments, a threshold is used to generate a positive result. In one aspect, an amplicon sequence must match the reference sequence at the threshold or above in order to know that the organism or virus being identified is indeed what the assay tests for. For example, if the threshold is 97% for a H3N2 influenza virus assay, then amplicon sequences that are less than 97% identical to the reference sequence can be ignored, as they represent influenza virus that is not H3N2 influenza virus and are a negative result on the assay. Sequences at 97% or greater similarity to the reference are a positive result for the H3N2 influenza virus assay. This may be particularly useful for certain assays where there is cross-reactivity of the primers with non-targets, but amplicon sequencing can distinguish targets from non-targets. In some embodiments, the threshold is about 90%, about 91%, about 92%, about 93%, about 94%, about 95%, about 96%, about 97%, about 98%, about 99% or about 100%. In example embodiments, the threshold is the maximum number of SNPs allowed in the amplicons compared to the reference sequence. In example embodiments, about 1, 2, 3, 4, 5, 6, 7, 8, 9, or 10 SNPs is the threshold.

In some embodiments, sample or biological sample may include a bodily tissue, fluid, or any other specimen that may be obtained from a living organism that may comprise additional living organisms. By way of example only, in some embodiments, sample or biological sample may include a specimen from a first organism (e.g., a human) that may further comprise an additional organism (e.g., bacteria, including pathogenic or non-pathogenic/commensal bacteria, viruses, parasites, fungi, including pathogenic or non-pathogenic fungi, etc.). In some embodiments of the invention, the additional organism may be separately cultured after isolation of the sample to provide additional starting materials for downstream analyses. In some embodiments, the sample or biological sample may comprise a direct portion of the additional, non-human organism and the host organism (e.g., a biopsy or sputum sample that contains human cells and bacteria).

With respect to use of the sample or biological sample, embodiments of the claimed methodology provide improvements compared to conventional methodologies. Specifically, conventional methodologies of identifying and characterizing microorganisms and/or viruses include the need for morphological identification and culture growth. As such, conventional methodologies may take an extended period of time to identify the microorganism and/or virus and may then require further time to identify whether the microorganism and/or virus possesses certain markers. Some embodiments of the invention can provide a user with information about any microorganisms and/or viruses present in a sample without the need for additional culturing because of the reliance of nucleic acid amplification and sequencing. In other words, direct extraction of nucleic acids coupled with amplification of the desired markers and downstream sequencing can reduce significantly the time required to obtain diagnostic and strain identifying information.

The invention may further comprise the step of sequencing the amplicon. Methods of sequencing include but need not be limited to any form of DNA sequencing including Sanger, next-generation sequencing, pyrosequencing, SOLiD sequencing, massively parallel sequencing, pooled, and barcoded DNA sequencing or any other sequencing method now known or yet to be disclosed.

In Sanger Sequencing, a single-stranded DNA template, a primer, a DNA polymerase, nucleotides and a label such as a radioactive label conjugated with the nucleotide base or a fluorescent label conjugated to the primer, and one chain terminator base comprising a dideoxynucleotide (ddATP, ddGTP, ddCTP, or ddTTP, are added to each of four reaction (one reaction for each of the chain terminator bases). The sequence may be determined by electrophoresis of the resulting strands. In dye terminator sequencing, each of the chain termination bases is labeled with a fluorescent label of a different wavelength that allows the sequencing to be performed in a single reaction.

In pyrosequencing, the addition of a base to a single-stranded template to be sequenced by a polymerase results in the release of a pyrophosphate upon nucleotide incorporation. An ATP sulfuryrlase enzyme converts pyrophosphate into ATP that in turn catalyzes the conversion of luciferin to oxyluciferin which results in the generation of visible light that is then detected by a camera or other sensor capable of capturing visible light.

In SOLID sequencing, the molecule to be sequenced is fragmented and used to prepare a population of clonal magnetic beads (in which each bead is conjugated to a plurality of copies of a single fragment) with an adaptor sequence and alternatively a barcode sequence. The beads are bound to a glass surface. Sequencing is then performed through 2-base encoding.

In massively parallel sequencing (i.e., next generation sequencing), randomly fragmented targeted nucleic acids and/or amplicons are attached to a surface. The fragments/amplicons are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

Nucleic acid sequences may be identified by the IUAPC letter code which is as follows: A-Adenine base; C-Cytosine base; G-guanine base; T or U-thymine or uracil base; I-inosine base. M-A or C; R-A or G; W-A or T; S-C or G; Y-C or T; K-G or T; V-A or C or G; H A or C or T; D-A or G or T; B-C or G or T; N or X-A or C or G or T. Note that T or U may be used interchangeably depending on whether the nucleic acid is DNA or RNA. A sequence having less than 60%, 70%, 80%, 90%, 95%, 99% or 100% identity to the identifying sequence may still be encompassed by the invention if it is capable of binding to its complimentary sequence and/or facilitating nucleic acid amplification of a desired target sequence. In some embodiments, as previously mentioned, the method may include the use of massively parallel sequencing, as detailed in U.S. Pat. Nos. 8,431,348 and 7,754,429, which are hereby incorporated by reference in their entirety.

Some embodiments of the invention comprise multiple steps and/or processes that are carried out to execute the universal tail indexing strategy to prepare amplicons corresponding to desired markers for sequencing. In some embodiments, one or more makers for a given sample or template can be selected, as described above. Some embodiments of the invention can be used in conjunction with an analysis of one or more markers (e.g., genes/alleles) associated with a particular phenotype (e.g., virulence).

After selection of the markers, marker-specific primers/oligonucleotides can be designed for the amplification of the markers to produce the desired amplicons, as detailed above. As is known in the art, a forward and a reverse marker-specific primer can be designed to amplify the marker from a nucleic acid sample. In some embodiments, the forward and reverse primers can be designed to produce an amplicon (e.g., some or all of the sequence of the marker) of a desired length. For example, the length of the amplicon may comprise approximately 50 base pairs (bp), 100 bp, 150 bp, 200 bp, 250 bp, 300 bp, 350 bp, 400 bp, 450 bp, 500 bp, 1,000 bp, or any size amplicon greater in size or therebetween.

As previously mentioned, some embodiments of the invention may include a multiplex PCR reaction. For example, marker-specific primers can be designed for multiple markers or multiple regions of the same marker such that multiple amplicons of between about 50 bp and 1,000 bp are being produced within a single PCR reaction vessel. In other words, the forward and reverse primers can be designed to function within a given set of temperature parameters such that more than one amplicon can be successfully amplified from a given template within a single PCR reaction mixture. As such, multiple amplicons can be prepared using the universal tail indexing strategy for sequencing preparation.

In some embodiments, the forward and reverse primers that have been designed for each of the markers can be modified to include a universal tail. For example, the universal tail sequences can be relatively or completely unique sequences of nucleotides that are coupled to the 5′ ends of some or all of the forward and reverse marker-specific primers. In some aspects, the universal tail sequences can be selected such that there is little to no overlap in sequence between portions of the markers that are being amplified and the universal tail sequences. Moreover, the universal tail sequences can comprise a length between ten and twenty nucleotides in length. In some embodiments, the universal tail sequences can be any other length, as desired by the user to meet the needs and requirements of the reaction. As such, the universal tail sequences can exhibit a relatively negligible impact on binding of the forward and reverse marker-specific primers to the template sequence to enable amplification. Moreover, as a result of being included on the 5′ end of the forward and reverse marker-specific primers, the universal tail sequences will form a portion of the resulting amplicons. In addition, in some aspects of the invention, the sequences selected for the universal tail sequences can be at least partially correlated with the chemical composition of the template nucleic acids. For example, in some aspects, the sequences selected for the universal tail sequences can be at least partially correlated with the G-C content of the organism from which the template is isolated.

In some aspects, some or all of the universal tail sequences can be at least partially unique. In some embodiments, each of the 5′ ends of all of the forward marker-specific primers within a given PCR assay mixture can comprise the same or a similar universal tail sequence (e.g., a first universal tail sequence or UT1). Similarly, each of the 5′ ends of all of the reverse marker-specific primers within the same PCR assay mixture can comprise a second universal tail sequence (UT2) that differs from the first universal tail sequence. As such, each respective sample from which a template sequence is used in the multiplex PCR assay will have two unique universal tail sequences. Accordingly, each forward and reverse marker-specific primer within a multiplex PCR mixture will include a unique universal tail sequence. For example, if the PCR includes 35 different samples, 35 universal tail sequences can be employed for the forward primers in each of the 35 unique reactions (i.e., not including technical replicates) and 35 universal tail sequences can be employed for the reverse primers in each of the 35 unique reactions (i.e., not including technical replicates). Overall, the forward and reverse marker-specific primers that each comprise the universal tail sequences can comprise a generally short length (e.g., 25-50 bp), which can facilitate simultaneous amplification of multiple targets in a single reaction.

In addition, some embodiments of the invention may comprise performing quantitative PCR to optimize the multiplex PCR assay. For example, after design of the forward and reverse marker-specific primers that each include a universal tail sequence, the contemplated multiplex PCR assays can be performed using quantitative PCR (e.g., using DNA as a template) to assess relative quantities of the amplicons produced. Accordingly, the sequence coverage of each amplicon is considered to be equal if the quantities of the amplicons produced by the multiplex quantitative PCR appear to be equal. If the quantities of the amplicons produced by the multiplex quantitative PCR do not appear to be equal, the forward and/or reverse marker-specific primers can be altered and re-optimized until adequate quantities of amplicons are produced.

After design and adequate optimization of the multiplex PCR assay comprising multiple forward and reverse marker-specific primers that each includes universal tail sequences, the multiplex PCR can be performed to obtain the amplicons associated with the above-described markers. In some embodiments, template that has been previously isolated from a sample can be used for the amplification of the amplicons. In some aspects, multiple PCR reaction replicates can be performed for each sample template and one or more control templates.

In some embodiments, after successful production of the amplicons during the multiplex PCR assay, the resulting amplicons can be further processed to provide sequencing-ready amplicons. For example, some embodiments of the invention may comprise an indexing extension step. In some aspects, the indexing extension step may comprise extending the optimized multiplex amplicons using a set of indexing and common primers that recognize the respective universal tail sequences used for the particular group of amplicons in a minimal cycle PCR assay (e.g., 5-10 total cycles). In particular, each multiplex set of amplicons to be sequenced can be extended with a different set of index oligonucleotides and common oligonucleotides that recognize UT1 and UT2, respectively. In some aspects, the index sequence of the index oligonucleotides can be custom designed to allow for the selection of an index sequence from potentially thousands of different index sequences.

After this step, the resulting products include a set of amplicons for each sample/template that comprise the same index and any necessary sequences that may be required for a particular sequencing platform (e.g., platform sequences associated with the ILLUMINA® Next Generation sequencing platform). Thereafter, the resulting extension-reaction products can be quantified, pooled, and sequenced using a desired platform. In some aspects, the inclusion of the universal tail sequences on the index and common primers can coincide with the use of genomic and index read primers in the mixture of sequencing primer reagents. For example, some embodiments of the invention are capable of pooling multiple amplicons with multiple indices in a single sequencing run to provide 40,000×-95,000× coverage across the amplicons. In other embodiments, the systems and methods associated with the invention can be configured to provide any level of sequencing coverage that is desirable to the user (e.g., higher or lower that the coverage levels discussed above). In some embodiments, after sequencing and generation of the sequence data, the resulting data can be demultiplexed and the sequence files can be aligned to the appropriate reference sequences for subsequent sequence analyses.

Embodiments of the invention offer additional advantages relative to conventional systems. For example, some embodiments of the invention comprise the use of PCR before sequencing such that only limited amounts of starting material are necessary and the starting material need not be of high quality (e.g., genomic DNA, crude DNA extracts, single stranded DNA, RNA, cDNA, etc.). In contrast, many conventional sample preparation systems may require relatively large amounts of starting material of relatively high quality, which can limit the use of these systems. Moreover, the inclusion of non-desirable template materials can also interfere in one or more downstream processes in conventional systems and methods. For example, if an investigation is being conducted that focuses on one or more organisms that may be associated with another organism (e.g., bacteria associated with a human); the sampling of the target organism may result in template contamination from the host organism.

In particular, in some aspects, obtaining samples from, on, or within a human may also result in the collection of human tissue. As such, when isolating the template, human nucleic acids may contaminate the viral template. Some embodiments of the invention are configured such that the contaminating template (e.g., from a human) would not interfere with downstream processes, including sequencing. For example, some embodiments of the invention operate such that only a limited amount of starting template (e.g., 500 femtograms or greater) can be used. Moreover, some embodiments are also configured such that the starting material (e.g., template contaminated with foreign nucleic acids) can still produce the required amplicons for sequencing in the presence of more than a 1,000-fold excess of contaminating template with no discernible inhibition of the multiplex PCR.

In certain aspects, the present invention provides an assay that works with as little as about 1 pg, about 900 fg, about 800 fg, about 700 fg, about 600 fg, about 500 fg, about 400 fg, about 300 fg, about 200 fg, or about 100 fg of viral RNA.

### Kits

The instant disclosure also provides kits containing agents of this disclosure for use in the methods of the present disclosure. Kits of the instant disclosure may include one or more primers of this disclosure. In some embodiments, the kits further include instructions for use in accordance with the methods of this disclosure. Kits may optionally provide additional components such as buffers and enzymes.

In certain aspects, the methods and kits of the present invention are used as a surveillance tool for H3N2 influenza virus (e.g., such as a healthcare facility). If the H3N2 influenza virus assays, methods, and/or kits produce positive results (i.e., detect the presence of the H3N2 influenza virus species) this result could be used be to limit the subsequent infection and transmission of the H3N2 influenza virus to another subject, patient, or health care worker.

In other aspects, the H3N2 influenza virus assays, methods, and/or kits disclosed herein are used as a diagnostic tool. Upon detection of a positive result in a subject, the subject is treated with an antiviral regimen.

### Methods of Treatment

In example embodiments, subjects positive for H3N2 influenza virus are treated with an antiviral drug. There are four FDA-approved antiviral drugs recommended by CDC to treat flu: oseltamivir phosphate (available as a generic version or under the trade name Tamiflu®), zanamivir (trade name Relenza®), peramivir (trade name Rapivab®), and baloxavir marboxil (trade name Xofluza®). In example embodiments, the antiviral drug is selected from the group consisting of oseltamivir phosphate, zanamivir, peramivir, and baloxavir marboxil.

Oseltamivir, sold under the brand name Tamiflu, is an antiviral medication used to treat and prevent influenza A and influenza B, viruses that cause the flu. Oseltamivir is a neuraminidase inhibitor, a competitive inhibitor of influenza's neuraminidase enzyme. The enzyme cleaves the sialic acid which is found on glycoproteins on the surface of human cells that helps new virions to exit the cell, preventing new viral particles from being released.

Zanamivir is a medication used to treat and prevent influenza caused by influenza A and influenza B viruses. It is a neuraminidase inhibitor.

Peramivir is a neuraminidase inhibitor approved to treat Type A and B influenza. Peramivir acts as a transition-state analogue inhibitor of influenza neuraminidase and thereby prevents new viruses from emerging from infected cells.

Baloxavir marboxil, sold under the brand name Xofluza, is an antiviral medication for treatment of influenza A and influenza B. Baloxavir marboxil is an influenza therapeutic agent, specifically, an enzyme inhibitor targeting the influenza virus' cap-dependent endonuclease activity, one of the activities of the virus polymerase complex. In particular, it inhibits a process known as cap snatching by which the virus derives short, capped primers from host cell RNA transcripts, which it then uses for polymerase-catalyzed synthesis of its needed viral mRNAs. A polymerase subunit binds to the host pre-mRNAs at their 5′ caps, then the polymerase's endonuclease activity catalyzes its cleavage “after 10-13 nucleotides”. As such, its mechanism is distinct from neuraminidase inhibitors such as oseltamivir and zanamivir.

Further embodiments are illustrated in the following Examples which are given for illustrative purposes only and are not intended to limit the scope of the invention.

### EXAMPLES

### Example 1—Detection of H3N2 Influenza Variants

At least four introductions of Influenza A occurred in the Northern Arizona community studied, one of which represents a rapid outbreak over a short period of time. Use of an amplicon primer panel approach proved to be a cost and time effective method to sequence complete H3N2 genomes.

TABLES 1 and 2 illustrate PCR and amplicon sequencing assays targeting the H3N2 genome. In TABLE 1, the universal tails added to the primers for amplicon sequencing are underlined. Universal tail sequences are ACCCAACTGAATGGAGC for forward read (SEQ ID NO: 173) and ACGCACTTGACTTGTCTTC for reverse read (SEQ ID NO: 174). The universal tail sequences (underlined) precede the assay-specific primer sequence, for example, in SEQ ID NOS: 1-86.

TABLE 1

| Assay | | | SEQ |
|---|---|---|---|
| component | Name | Sequence | ID NO: |
| Amplicon | FluA_2022_Seg1_1L | ACCCAACTGAATGGAGCTATATTCAGTAT | 1 |
| sequencing | _NEW_UT | GGAAAGAATAAAAGA | |
| primer | | | |
| Amplicon | InfluenzaA_2022_2 | ACCCAACTGAATGGAGCACGGTCCATTA | 2 |
| sequencing | _LEFT UT | CCCAAAAGTGT | |
| primer | | | |
| Amplicon | InfluenzaA_2022_3 | ACCCAACTGAATGGAGCGTGCGGAAAAC | 3 |
| sequencing | _LEFT_UT | AAGATTTCTCCC | |
| primer | | | |
| Amplicon | InfluenzaA_2022_4 | ACCCAACTGAATGGAGCTCATCTTTCAGC | 4 |
| sequencing | _LEFT UT | TTTGGTGGCT | |
| primer | | | |
| Amplicon | InfluenzaA 2022_5 | ACCCAACTGAATGGAGCCAGAGCAAATC | 5 |
| sequencing | _LEFT_UT | AGCGGTTGAAC | |
| primer | | | |
| Amplicon | InfluenzaA_2022_6 | ACCCAACTGAATGGAGCATGATGTGGGA | 6 |
| sequencing | _LEFT_UT | GATTAACGGCC | |
| primer | | | |
| Amplicon | InfluenzaA_2022_7 | ACCCAACTGAATGGAGCCCACCGAAGCA | 7 |
| sequencing | _LEFT_UT | AAGCAGAATG | |
| primer | | | |
| Amplicon | FluA_2022_Seg2_1L | ACCCAACTGAATGGAGCCAAGACCATTT | 8 |
| sequencing | _NEW_UT | GAATGGATGTCAA | |
| primer | | | |
| Amplicon | InfluenzaA_2022_9 | ACCCAACTGAATGGAGCTTGATGGACCA | 9 |
| sequencing | _LEFT_UT | CTACCTGAGGA | |
| primer | | | |
| Amplicon | InfluenzaA_2022_10 | ACCCAACTGAATGGAGCTCAATGGATAA | 10 |
| sequencing | _LEFT_UT | GGAGGAAATGGAGA | |
| primer | | | |
| Amplicon | InfluenzaA_2022_11 | ACCCAACTGAATGGAGCCCAAACTGGCA | 11 |
| sequencing | _LEFT_UT | AATGTTGTGAGA | |
| primer | | | |
| Amplicon | InfluenzaA_2022_12 | ACCCAACTGAATGGAGCCAATGAATCAA | 12 |
| sequencing | _LEFT_UT | CAAGGAAGAAAATTGAGA | |
| primer | | | |
| Amplicon | InfluenzaA_2022_13 | ACCCAACTGAATGGAGCACAGGGACATT | 13 |
| sequencing | _LEFT_UT | TGAATTCACTAGCT | |
| primer | | | |
| Amplicon | InfluenzaA_2022_14 | ACCCAACTGAATGGAGCCAGGATTATTG | 14 |
| sequencing | _LEFT_UT | GTATCAGATGGGGG | |
| primer | | | |
| Amplicon | InfluenzaA_2022_15 | ACCCAACTGAATGGAGCACTTGTTCGAG | 15 |
| sequencing | _LEFT_UT | AAATTTTTCCCTAGT | |
| primer | | | |
| Amplicon | FluA_2022_Seg3_1L | ACCCAACTGAATGGAGCGCTTCAACCCG | 16 |
| sequencing | _NEW_UT | ATGATTGTCGA | |
| primer | | | |
| Amplicon | InfluenzaA_2022_17 | ACCCAACTGAATGGAGCAAATCAGAGAA | 17 |
| sequencing | _LEFT_UT | CACACACATTCACA | |
| primer | | | |
| Amplicon | InfluenzaA_2022_18 | ACCCAACTGAATGGAGCGGGCAAGCTTT | 18 |
| sequencing | _LEFT_UT | CTCAAATGTCC | |
| primer | | | |
| Amplicon | InfluenzaA_2022_19 | ACCCAACTGAATGGAGCTGAAAATGAGG | 19 |
| sequencing | _LEFT_UT | AGAAGATTCCAAGAAC | |
| primer | | | |
| Amplicon | InfluenzaA_2022_20 | ACCCAACTGAATGGAGCGTCCCATTGCA | 20 |
| sequencing | _LEFT_UT | GAGCTACTGAA | |
| primer | | | |
| Amplicon | InfluenzaA_2022_21 | ACCCAACTGAATGGAGCGCCATAGGCCA | 21 |
| sequencing | _LEFT_UT | AATTTCAAGGC | |
| primer | | | |
| Amplicon | InfluenzaA_2022_22 | ACCCAACTGAATGGAGCGCAGAGTCAAG | 22 |
| sequencing | _LEFT_UT | AAAACTGCTCC | |
| primer | | | |
| Amplicon | FluA_2022_Seg4_1L | ACCCAACTGAATGGAGCTAATGTCTATTA | 23 |
| sequencing | _NEW_UT | ACCATGAAGACTATC | |
| primer | | | |
| Amplicon | InfluenzaA_2022_24 | ACCCAACTGAATGGAGCTCCGGCACACT | 24 |
| sequencing | _LEFT_UT | GGAGTTTAAAA | |
| primer | | | |
| Amplicon | InfluenzaA_2022_25 | ACCCAACTGAATGGAGCTCCCTAGCAGA | 25 |
| sequencing | _LEFT_UT | ATAAGCATCTATTGG | |
| primer | | | |
| Amplicon | InfluenzaA_2022_26 | ACCCAACTGAATGGAGCACAAACCAGAG | 26 |
| sequencing | _LEFT_UT | GCATATTTGGC | |
| primer | | | |
| Amplicon | InfluenzaA_2022_27 | ACCCAACTGAATGGAGCTTGCCCTGGAG | 27 |
| sequencing | _LEFT_UT | AACCAACATAC | |
| primer | | | |
| Amplicon | FluA_2022_Seg5_1L | ACCCAACTGAATGGAGCGTAGATAATCA | 28 |
| sequencing | _NEW_UT | CTCACTGAGTGA | |
| primer | | | |
| Amplicon | InfluenzaA_2022_29 | ACCCAACTGAATGGAGCTGATGAAAGAA | 29 |
| sequencing | _LEFT_UT | GAAATAAATACCTGGAAGA | |
| primer | | | |
| Amplicon | InfluenzaA_2022_30 | ACCCAACTGAATGGAGCAGGTGCTGCAG | 30 |
| sequencing | _LEFT_UT | TCAAAGGAATC | |
| primer | | | |
| Amplicon | InfluenzaA_2022_31 | ACCCAACTGAATGGAGCCCTGCAGTATCC | 31 |
| sequencing | _LEFT_UT | AGTGGTTACG | |
| primer | | | |
| Amplicon | InfluenzaA_2022_32 | ACCCAACTGAATGGAGCTCAACTAGAGG | 32 |
| sequencing | _LEFT_UT | AGTACAAATTGCTTCA | |
| primer | | | |
| Amplicon | FluA_2022_Seg6_1L | ACCCAACTGAATGGAGCCGATTGGCTCTG | 33 |
| sequencing | _NEW_UT | TTTCTCTCACA | |
| primer | | | |
| Amplicon | InfluenzaA_2022_34 | ACCCAACTGAATGGAGCAGTGTATTTGAC | 34 |
| sequencing | _LEFT_UT | CAACACCACCATA | |
| primer | | | |
| Amplicon | InfluenzaA_2022_35 | ACCCAACTGAATGGAGCGTGTTCCTTTCC | 35 |
| sequencing | _LEFT_UT | ATCTGGGGAC | |
| primer | | | |
| Amplicon | InfluenzaA_2022_36 | ACCCAACTGAATGGAGCGAAGTGCTCAG | 36 |
| sequencing | _LEFT_UT | CATGTCGAAGA | |
| primer | | | |
| Amplicon | InfluenzaA_2022_37 | ACCCAACTGAATGGAGCTCAAAGTCGTT | 37 |
| sequencing | _LEFT_UT | GAAGGCTGGTC | |
| primer | | | |
| Amplicon | FluA_2022_Seg7_1L | ACCCAACTGAATGGAGCTAGATATTGAA | 38 |
| sequencing | _NEW_UT | AGATGATCTTACCG | |
| primer | | | |
| Amplicon | InfluenzaA_2022_39 | ACCCAACTGAATGGAGCTGGAGACCCAA | 39 |
| sequencing | _LEFT_UT | ATAACATGGACA | |
| primer | | | |
| Amplicon | InfluenzaA_2022_40 | ACCCAACTGAATGGAGCGCAAATGGCTG | 40 |
| sequencing | _LEFT_UT | GCTCAAGTGA | |
| primer | | | |
| Amplicon | FluA_2022_Seg8_1L | ACCCAACTGAATGGAGCGTGTATAAGAG | 41 |
| sequencing | _NEW_UT | ACATAATGGATTCCA | |
| primer | | | |
| Amplicon | InfluenzaA_2022_42 | ACCCAACTGAATGGAGCGCAAATCGTAG | 42 |
| sequencing | _LEFT_UT | AAAAGATTCTGAAAGG | |
| primer | | | |
| Amplicon | InfluenzaA_2022_43 | ACCCAACTGAATGGAGCATTGCTAAGGG | 43 |
| sequencing | _LEFT_UT | CTTTCACCGAA | |
| primer | | | |
| Amplicon | InfluenzaA_2022_1 | ACGCACTTGACTTGTCTTCAAAATGGACA | 44 |
| sequencing | _RIGHT_UT | GGGCCAAAGGTT | |
| primer | | | |
| Amplicon | InfluenzaA_2022_2_ | ACGCACTTGACTTGTCTTCTTCCCAACAC | 45 |
| sequencing | _RIGHT_UT | GTTCCTTGAGTC | |
| primer | | | |
| Amplicon | InfluenzaA_2022_3_ | ACGCACTTGACTTGTCTTCTGCCTGTAAG | 46 |
| sequencing | _RIGHT_UT | AACCTCTTCTTCTC | |
| primer | | | |
| Amplicon | InfluenzaA_2022_4 | ACGCACTTGACTTGTCTTCCGATGTGTTC | 47 |
| sequencing | _RIGHT_UT | AACTCCCCAATTT | |
| primer | | | |
| Amplicon | InfluenzaA_2022_5 | ACGCACTTGACTTGTCTTCTTGACAGCTT | 48 |
| sequencing | _RIGHT_UT | CCCAATTCCTGA | |
| primer | | | |
| Amplicon | InfluenzaA_2022_6 | ACGCACTTGACTTGTCTTCAGCCTTTTAG | 49 |
| sequencing | _RIGHT_UT | TGGTCTTGTTGTAGT | |
| primer | | | |
| Amplicon | FluA_2022_Seg1_7R | ACGCACTTGACTTGTCTTCTTTTTAAACTA | 50 |
| sequencing | _NEW_UT | TTCAGTATTAATTGATGG | |
| primer | | | |
| Amplicon | InfluenzaA_2022_8 | ACGCACTTGACTTGTCTTCGGATTCTTCA | 51 |
| sequencing | _RIGHT_UT | AGAAAGGCCATAGC | |
| primer | | | |
| Amplicon | InfluenzaA_2022_9 | ACGCACTTGACTTGTCTTCGTTCTTTGTGT | 52 |
| sequencing | _RIGHT_UT | GACCATTTTCTTGG | |
| primer | | | |
| Amplicon | InfluenzaA_2022_10 | ACGCACTTGACTTGTCTTCTCCACTTAGT | 53 |
| sequencing | _RIGHT_UT | GTTGTCTCCAGTG | |
| primer | | | |
| Amplicon | InfluenzaA_2022_11 | ACGCACTTGACTTGTCTTCACATGCCCAT | 54 |
| sequencing | _RIGHT_UT | CATCATTCCAGG | |
| primer | | | |
| Amplicon | InfluenzaA_2022_12 | ACGCACTTGACTTGTCTTCTCGTTTATTCC | 55 |
| sequencing | _RIGHT_UT | AGACACTCCAAAAC | |
| primer | | | |
| Amplicon | InfluenzaA_2022_13 | ACGCACTTGACTTGTCTTCCCCCGATAAT | 56 |
| sequencing | _RIGHT_UT | TGTCGTCCATCA | |
| primer | | | |
| Amplicon | FluA_2022_Seg2_7R | ACGCACTTGACTTGTCTTCCATTTTTTCAT | 57 |
| sequencing | _NEW_UT | GAAGGACAAGCTAAA | |
| primer | | | |
| Amplicon | InfluenzaA_2022_15 | ACGCACTTGACTTGTCTTCTCGCCTTGTTC | 58 |
| sequencing | _RIGHT_UT | ATTGATGAAATGG | |
| primer | | | |
| Amplicon | InfluenzaA_2022_16 | ACGCACTTGACTTGTCTTCTCCTCGTCGA | 59 |
| sequencing | _RIGHT_UT | GAGTGTAGTCTG | |
| primer | | | |
| Amplicon | InfluenzaA_2022_17 | ACGCACTTGACTTGTCTTCTCCACTGGGA | 60 |
| sequencing | _RIGHT_UT | AGTTTGATTGGT | |
| primer | | | |
| Amplicon | InfluenzaA_2022_18 | ACGCACTTGACTTGTCTTCTCTACCTTCTC | 61 |
| sequencing | _RIGHT_UT | TGGTGCCATGT | |
| primer | | | |
| Amplicon | InfluenzaA_2022_19 | ACGCACTTGACTTGTCTTCAGTTGAAAAT | 62 |
| sequencing | _RIGHT_UT | CGTCCATTGCCG | |
| primer | | | |
| Amplicon | InfluenzaA_2022_20 | ACGCACTTGACTTGTCTTCCAACGTCTCA | 63 |
| sequencing | _RIGHT_UT | TCTCCATTCCCC | |
| primer | | | |
| Amplicon | FluA_2022_Seg3_7R | ACGCACTTGACTTGTCTTCTAGCTTTTTTG | 64 |
| sequencing | _NEW_UT | GACAGTACGGAT | |
| primer | | | |
| Amplicon | InfluenzaA_2022_22 | ACGCACTTGACTTGTCTTCTCTGAACCAA | 65 |
| sequencing | _RIGHT_UT | CTCAGTAGCATTAGT | |
| primer | | | |
| Amplicon | InfluenzaA_2022_23 | ACGCACTTGACTTGTCTTCAAAACTACTA | 66 |
| sequencing | _RIGHT_UT | CTAGATCCCCTTATGCA | |
| primer | | | |
| Amplicon | InfluenzaA_2022_24 | ACGCACTTGACTTGTCTTCGGAGCAATTA | 67 |
| sequencing | _RIGHT_UT | GATTCCCTGTGCT | |
| primer | | | |
| Amplicon | InfluenzaA_2022_25 | ACGCACTTGACTTGTCTTCTGATGCCTGA | 68 |
| sequencing | _RIGHT_UT | AACCGTACCAAC | |
| primer | | | |
| Amplicon | InfluenzaA_2022_26 | ACGCACTTGACTTGTCTTCTCAGCATTTTC | 69 |
| sequencing | _RIGHT_UT | CCTCAGTTGCT | |
| primer | | | |
| Amplicon | FluA_2022_Seg4_5R | ACGCACTTGACTTGTCTTCGTTTTTAATTA | 70 |
| sequencing | _NEW_UT | ATGCACTCAAATGC | |
| primer | | | |
| Amplicon | InfluenzaA_2022_28 | ACGCACTTGACTTGTCTTCGACTCTTCTGT | 71 |
| sequencing | _RIGHT_UT | ATATGGGCCCC | |
| primer | | | |
| Amplicon | InfluenzaA_2022_29 | ACGCACTTGACTTGTCTTCAATTTCGATC | 72 |
| sequencing | _RIGHT_UT | GTTGATCCCCCG | |
| primer | | | |
| Amplicon | InfluenzaA_2022_30 | ACGCACTTGACTTGTCTTCGGCTATTTTG | 73 |
| sequencing | _RIGHT_UT | AAGTAGTTTGAAAGGGT | |
| primer | | | |
| Amplicon | InfluenzaA_2022_31 | ACGCACTTGACTTGTCTTCAAAACGTAGG | 74 |
| sequencing | _RIGHT_UT | TTGCACACTGGT | |
| primer | | | |
| Amplicon | FluA_2022_Seg5_5R | ACGCACTTGACTTGTCTTCGGTATTGTTTC | 75 |
| sequencing | _NEW_UT | CTTAATTGTCG | |
| primer | | | |
| Amplicon | InfluenzaA_2022_33 | ACGCACTTGACTTGTCTTCCCACATTGCG | 76 |
| sequencing | _RIGHT_UT | GTTTTGACCAAT | |
| primer | | | |
| Amplicon | InfluenzaA_2022_34 | ACGCACTTGACTTGTCTTCCCCGTTATAC | 77 |
| sequencing | _RIGHT_UT | AAACATGCAGCC | |
| primer | | | |
| Amplicon | InfluenzaA_2022_35 | ACGCACTTGACTTGTCTTCGGATCCTTTC | 78 |
| sequencing | _RIGHT_UT | CAGTTGTCTCTGC | |
| primer | | | |
| Amplicon | FluA_2022_Seg6_4R | ACGCACTTGACTTGTCTTCGAGTTTGTTTC | 79 |
| sequencing | _NEW_UT | TATAATTGCGAAAG | |
| primer | | | |
| Amplicon | InfluenzaA_2022_37 | ACGCACTTGACTTGTCTTCATCTTCAAGT | 80 |
| sequencing | _RIGHT_UT | CTCTGCGCGATC | |
| primer | | | |
| Amplicon | InfluenzaA_2022_38 | ACGCACTTGACTTGTCTTCTGAGAGCTAT | 81 |
| sequencing | _RIGHT_UT | TTCTTTGGCCCC | |
| primer | | | |
| Amplicon | InfluenzaA_2022_39 | ACGCACTTGACTTGTCTTCACTAGGATGA | 82 |
| sequencing | _RIGHT_UT | GTCCCAATGGCT | |
| primer | | | |
| Amplicon | FluA_2022_Seg7_3R | ACGCACTTGACTTGTCTTCGTAGTTTTTTA | 83 |
| sequencing | _NEW_UT | CTCCAATTCTATGC | |
| primer | | | |
| Amplicon | InfluenzaA_2022_41 | ACGCACTTGACTTGTCTTCAGTCATGTCA | 84 |
| sequencing | _RIGHT_UT | GTTATGTATCGCGA | |
| primer | | | |
| Amplicon | InfluenzaA_2022_42 | ACGCACTTGACTTGTCTTCTGTAGATTTTT | 85 |
| sequencing | _RIGHT_UT | AGAGACTCGAACTGTGT | |
| primer | | | |
| Amplicon | FluA_2022_Seg8_3R | ACGCACTTGACTTGTCTTCGTGTGTTTTTT | 86 |
| sequencing | _NEW_UT | ATCATTAAATAAGCTG | |
| primer | | | |

TABLE 2

| Assay | | | SEQ |
|---|---|---|---|
| component | Name | Sequence | ID NO: |
| PCR Primer | FluA_2022_Seg1_1L | TATATTCAGTATGGAAAGAATAAAAGA | 87 |
| | _NEW | | |
| PCR Primer | InfluenzaA_2022_2 | AAAATGGACAGGGCCAAAGGTT | 88 |
| | _LEFT | | |
| PCR Primer | InfluenzaA_2022_3 | ACGGTCCATTACCCAAAAGTGT | 89 |
| | _LEFT | | |
| PCR Primer | InfluenzaA_2022_4 | TTCCCAACACGTTCCTTGAGTC | 90 |
| | _LEFT | | |
| PCR Primer | InfluenzaA_2022_5 | GTGCGGAAAACAAGATTTCTCCC | 91 |
| | _LEFT | | |
| PCR Primer | InfluenzaA_2022_6 | TGCCTGTAAGAACCTCTTCTTCTC | 92 |
| | _LEFT | | |
| PCR Primer | InfluenzaA_2022_7 | TCATCTTTCAGCTTTGGTGGCT | 93 |
| | _LEFT | | |
| PCR Primer | FluA_2022_Seg2_1L | CAAGACCATTTGAATGGATGTCAA | 94 |
| | _NEW | | |
| PCR Primer | InfluenzaA_2022_9 | CAGAGCAAATCAGCGGTTGAAC | 95 |
| | _LEFT | | |
| PCR Primer | InfluenzaA_2022_10 | TTGACAGCTTCCCAATTCCTGA | 96 |
| | _LEFT | | |
| PCR Primer | InfluenzaA_2022_11 | ATGATGTGGGAGATTAACGGCC | 97 |
| | _LEFT | | |
| PCR Primer | InfluenzaA_2022_12 | AGCCTTTTAGTGGTCTTGTTGTAGT | 98 |
| | _LEFT | | |
| PCR Primer | InfluenzaA_2022_13 | CCACCGAAGCAAAGCAGAATG | 99 |
| | _LEFT | | |
| PCR Primer | InfluenzaA_2022_14 | AGCATTAATTGATGGCCATCCGA | 100 |
| | _LEFT | | |
| PCR Primer | InfluenzaA_2022_15 | CGTGGTGTTGGTAATGAAACGG | 101 |
| | _LEFT | | |
| PCR Primer | FluA_2022_Seg3_1L | GCTTCAACCCGATGATTGTCGA | 102 |
| | _NEW | | |
| PCR Primer | InfluenzaA_2022_17 | TTGATGGACCACTACCTGAGGA | 103 |
| | _LEFT | | |
| PCR Primer | InfluenzaA_2022_18 | GTTCTTTGTGTGACCATTTTCTTGG | 104 |
| | _LEFT | | |
| PCR Primer | InfluenzaA_2022_19 | TCAATGGATAAGGAGGAAATGGAGA | 105 |
| | _LEFT | | |
| PCR Primer | InfluenzaA_2022_20 | TCCACTTAGTGTTGTCTCCAGTG | 106 |
| | _LEFT | | |
| PCR Primer | InfluenzaA_2022_21 | CCAAACTGGCAAATGTTGTGAGA | 107 |
| | _LEFT | | |
| PCR Primer | InfluenzaA_2022_22 | ACATGCCCATCATCATTCCAGG | 108 |
| | _LEFT | | |
| PCR Primer | FluA_2022_Seg4_1L | TAATGTCTATTAACCATGAAGACTATC | 109 |
| | _NEW | | |
| PCR Primer | InfluenzaA_2022_24 | TCGTTTATTCCAGACACTCCAAAAC | 110 |
| | _LEFT | | |
| PCR Primer | InfluenzaA_2022_25 | ACAGGGACATTTGAATTCACTAGCT | 111 |
| | _LEFT | | |
| PCR Primer | InfluenzaA_2022_26 | CCCCGATAATTGTCGTCCATCA | 112 |
| | _LEFT | | |
| PCR Primer | InfluenzaA_2022_27 | CAGGATTATTGGTATCAGATGGGGG | 113 |
| | _LEFT | | |
| PCR Primer | FluA_2022_Seg5_1L | GTAGATAATCACTCACTGAGTGA | 114 |
| | _NEW | | |
| PCR Primer | InfluenzaA_2022_29 | ACTTGTTCGAGAAATTTTTCCCTAGT | 115 |
| | _LEFT | | |
| PCR Primer | InfluenzaA_2022_30 | TCGCCTTGTTCATTGATGAAATGG | 116 |
| | _LEFT | | |
| PCR Primer | InfluenzaA_2022_31 | TTGAAACCAACAAATTTGCAGCAA | 117 |
| | _LEFT | | |
| PCR Primer | InfluenzaA_2022_32 | TCCTCGTCGAGAGTGTAGTCTG | 118 |
| | _LEFT | | |
| PCR Primer | FluA_2022_Seg6_1L | CGATTGGCTCTGTTTCTCTCACA | 119 |
| | _NEW | | |
| PCR Primer | InfluenzaA_2022_34 | TCCACTGGGAAGTTTGATTGGT | 120 |
| | _LEFT | | |
| PCR Primer | InfluenzaA_2022_35 | GGGCAAGCTTTCTCAAATGTCC | 121 |
| | _LEFT | | |
| PCR Primer | InfluenzaA_2022_36 | TCTACCTTCTCTGGTGCCATGT | 122 |
| | _LEFT | | |
| PCR Primer | InfluenzaA_2022_37 | TGAAAATGAGGAGAAGATTCCAAGAAC | 123 |
| | _LEFT | | |
| PCR Primer | FluA_2022_Seg7_1L | TAGATATTGAAAGATGATCTTACCG | 124 |
| | _NEW | | |
| PCR Primer | InfluenzaA_2022_39 | GTCCCATTGCAGAGCTACTGAA | 125 |
| | _LEFT | | |
| PCR Primer | InfluenzaA_2022_40 | CAACGTCTCATCTCCATTCCCC | 126 |
| | _LEFT | | |
| PCR Primer | FluA_2022_Seg8_1L | GTGTATAAGAGACATAATGGATTCCA | 127 |
| | _NEW | | |
| PCR Primer | InfluenzaA_2022_42 | CCTCAATTGCTTCATATAGCCCCC | 128 |
| | _LEFT | | |
| PCR Primer | InfluenzaA_2022_43 | GCAGAGTCAAGAAAACTGCTCC | 129 |
| | _LEFT | | |
| PCR Primer | InfluenzaA_2022_1 | TCTGAACCAACTCAGTAGCATTAGT | 130 |
| | _RIGHT | | |
| PCR Primer | InfluenzaA_2022_2 | CACCATGCAGTACCAAACGGAA | 131 |
| | _RIGHT | | |
| PCR Primer | InfluenzaA_2022_3 | AAAACTACTACTAGATCCCCTTATGCA | 132 |
| | _RIGHT | | |
| PCR Primer | InfluenzaA_2022_4 | TCCGGCACACTGGAGTTTAAAA | 133 |
| | _RIGHT | | |
| PCR Primer | InfluenzaA_2022_5 | GGAGCAATTAGATTCCCTGTGCT | 134 |
| | _RIGHT | | |
| PCR Primer | InfluenzaA_2022_6 | TCCCTAGCAGAATAAGCATCTATTGG | 135 |
| | _RIGHT | | |
| PCR Primer | FluA_2022_Seg1_7R | TTTTTAAACTATTCAGTATTAATTGATGG | 136 |
| | _NEW | | |
| PCR Primer | InfluenzaA_2022_8 | ACAAACCAGAGGCATATTTGGC | 137 |
| | _RIGHT | | |
| PCR Primer | InfluenzaA_2022_9 | TCAGCATTTTCCCTCAGTTGCT | 138 |
| | _RIGHT | | |
| PCR Primer | InfluenzaA_2022_10 | TTGCCCTGGAGAACCAACATAC | 139 |
| | _RIGHT | | |
| PCR Primer | InfluenzaA_2022_11 | ACTCAGTGAGTGATTATCTACGTTTTTAA | 140 |
| | _RIGHT | | |
| PCR Primer | InfluenzaA_2022_12 | TTGTTGGGGTTCATCATGTGGG | 141 |
| | _RIGHT | | |
| PCR Primer | InfluenzaA_2022_13 | GACTCTTCTGTATATGGGCCCC | 142 |
| | _RIGHT | | |
| PCR Primer | FluA_2022_Seg2_7R | CATTTTTTCATGAAGGACAAGCTAAA | 143 |
| | _NEW | | |
| PCR Primer | InfluenzaA_2022_15 | AATTTCGATCGTTGATCCCCCG | 144 |
| | _RIGHT | | |
| PCR Primer | InfluenzaA_2022_16 | AGGTGCTGCAGTCAAAGGAATC | 145 |
| | _RIGHT | | |
| PCR Primer | InfluenzaA_2022_17 | GGCTATTTTGAAGTAGTTTGAAAGGGT | 146 |
| | _RIGHT | | |
| PCR Primer | InfluenzaA_2022_18 | CCTGCAGTATCCAGTGGTTACG | 147 |
| | _RIGHT | | |
| PCR Primer | InfluenzaA_2022_19 | AAAACGTAGGTTGCACACTGGT | 148 |
| | _RIGHT | | |
| PCR Primer | InfluenzaA_2022_20 | TCAACTAGAGGAGTACAAATTGCTTCA | 149 |
| | _RIGHT | | |
| PCR Primer | FluA_2022_Seg3_7R | TAGCTTTTTTGGACAGTACGGAT | 150 |
| | _NEW | | |
| PCR Primer | InfluenzaA_2022_22 | TTCGAGCTCTCAGACGAGAAGG | 151 |
| | _RIGHT | | |
| PCR Primer | InfluenzaA_2022_23 | CCACATTGCGGTTTTGACCAAT | 152 |
| | _RIGHT | | |
| PCR Primer | InfluenzaA_2022_24 | AGTGTATTTGACCAACACCACCATA | 153 |
| | _RIGHT | | |
| PCR Primer | InfluenzaA_2022_25 | CCCGTTATACAAACATGCAGCC | 154 |
| | _RIGHT | | |
| PCR Primer | InfluenzaA_2022_26 | GTGTTCCTTTCCATCTGGGGAC | 155 |
| | _RIGHT | | |
| PCR Primer | FluA_2022_Seg4_5R | GTTTTTAATTAATGCACTCAAATGC | 156 |
| | _NEW | | |
| PCR Primer | InfluenzaA_2022_28 | GAAGTGCTCAGCATGTCGAAGA | 157 |
| | _RIGHT | | |
| PCR Primer | InfluenzaA_2022_29 | GGACCTATCGCCTCTGTCAACT | 158 |
| | _RIGHT | | |
| PCR Primer | InfluenzaA_2022_30 | TCAAAGTCGTTGAAGGCTGGTC | 159 |
| | _RIGHT | | |
| PCR Primer | InfluenzaA_2022_31 | ATCTTCAAGTCTCTGCGCGATC | 160 |
| | _RIGHT | | |
| PCR Primer | FluA_2022_Seg5_5R | GGTATTGTTTCCTTAATTGTCG | 161 |
| | _NEW | | |
| PCR Primer | InfluenzaA_2022_33 | TGAGAGCTATTTCTTTGGCCCC | 162 |
| | _RIGHT | | |
| PCR Primer | InfluenzaA_2022_34 | TGGAGACCCAAATAACATGGACA | 163 |
| | _RIGHT | | |
| PCR Primer | InfluenzaA_2022_35 | ACTAGGATGAGTCCCAATGGCT | 164 |
| | _RIGHT | | |
| PCR Primer | FluA_2022_Seg6_4R | GAGTTTGTTTCTATAATTGCGAAAG | 165 |
| | _NEW | | |
| PCR Primer | InfluenzaA_2022_37 | TGCTGACAAAATGACTGTCGTC | 166 |
| | _RIGHT | | |
| PCR Primer | InfluenzaA_2022_38 | ACGGAAGGTGTACCTGAGTCTA | 167 |
| | _RIGHT | | |
| PCR Primer | InfluenzaA_2022_39 | AGTCATGTCAGTTATGTATCGCGA | 168 |
| | _RIGHT | | |
| PCR Primer | FluA_2022_Seg7_3R | GTAGTTTTTTACTCCAATTCTATGC | 169 |
| | _NEW | | |
| PCR Primer | InfluenzaA_2022_41 | TGTAGATTTTTAGAGACTCGAACTGTGT | 170 |
| | _RIGHT | | |
| PCR Primer | InfluenzaA_2022_42 | ATTGCTAAGGGCTTTCACCGAA | 171 |
| | _RIGHT | | |
| PCR Primer | FluA_2022_Seg8_3R | GTGTGTTTTTTATCATTAAATAAGCTG | 172 |
| | _NEW | | |

TABLE 3

| Assay | | | SEQ |
|---|---|---|---|
| component | Name | Sequence | ID NO: |
| PCR | Universal | ACCCAACT | 173 |
| Primer | Tail 1 | GAATGGAGC | |
| PCR | Universal | ACGCACTTG | 174 |
| Primer | Tail 2 | ACTTGTCTTC | |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

Many additional implementations are possible. Further implementations are within the CLAIMS. In places where the description above refers to particular methods, it should be readily apparent that a number of modifications may be made without departing from the spirit thereof and that these methods may be applied to other implementations disclosed or undisclosed. The presently disclosed methods are, therefore, to be considered in all respects as illustrative and not restrictive.

Various modifications and variations of the described methods, compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of detecting an H3N2 influenza virus in a sample, comprising:
producing one or more amplicons by amplifying one or more nucleic acid segments from the sample using:
(a) at least one forward primer selected from the group consisting of SEQ ID NOS: 1-43, and
(b) at least one reverse primer selected from the group consisting of SEQ ID NOS: 44-86; and
sequencing the one or more amplicons to detect the presence of the H3N2 influenza virus in the sample.

2. The method of claim 1, wherein the sample is an environmental biological sample.

3. The method of claim 1, wherein the sample is from a subject.

4. The method of claim 3, wherein the sample is obtained from an animal subject.

5. The method of claim 3, wherein the sample is obtained from a human subject.

6. The method of claim 1, wherein the sequencing is next-generation sequencing (NGS).

7. The method of claim 1, wherein the presence of the H3N2 influenza virus is detected in the sample if the amplicon sequences are greater than or equal to 90% identical to an H3N2 influenza virus reference sequence.

8. The method of claim 1, wherein the presence of the H3N2 influenza virus is detected in the sample if the amplicon sequences are greater than or equal to 95% identical to an H3N2 influenza virus reference sequence.

9. The method of claim 1, wherein the presence of the H3N2 influenza virus is detected in the sample if the amplicon sequences include 10 or less single nucleotide polymorphisms (SNPs) as compared to an H3N2 influenza virus reference sequence.

10. The method of claim 1, wherein if H3N2 influenza virus is detected in the sample the subject is treated with an antiviral treatment.

11. The method of claim 10, wherein the antiviral treatment is selected from the group consisting of oseltamivir phosphate, zanamivir, peramivir, and baloxavir marboxil.

12. A method of detecting the presence of H3N2 influenza virus in a sample, comprising:
performing real-time polymerase chain reaction (PCR) to amplify nucleic acid segments from the sample, wherein the amplifying step includes using:
(a) at least one forward primer selected from the group consisting of SEQ ID NOS: 87-129, and
(b) at least one reverse primer selected from the group consisting of SEQ ID NOS: 130-172; and
detecting the presence of H3N2 influenza virus in the sample by comparing the amplification results to a control value.

13. The method of claim 12, wherein H3N2 influenza virus is detected in the sample if the amplification reaches a threshold value at an earlier cycle than background amplification or a negative control value.

14. The method of claim 12, wherein amplification is detected by fluorescence of a detectably labeled probe that is specific to a sequence within an amplicon obtained by amplification with the forward and reverse primer during real-time PCR.

15. The method of claim 12, wherein the sample is an environmental biological sample.

16. The method of claim 12, wherein the sample is from a subject.

17. The method of claim 16, wherein the sample is obtained from an animal subject.

18. The method of claim 16, wherein the sample is obtained from a human subject.

19. The method of claim 12, wherein if H3N2 influenza virus is detected in the sample the subject is treated with an antiviral treatment.

20. The method of claim 19, wherein the antiviral treatment is selected from the group consisting of oseltamivir phosphate, zanamivir, peramivir, and baloxavir marboxil.
